(19) United States
(12) Patent Application Publication
(10) Pub. No.: US 2025/0160366 A1
(43) Pub. Date: May 22, 2025

(54) MEANS AND METHODS FOR MODULATING SECONDARY METABOLITE IN AN ANIMAL

(71) Applicant: DSM IP Assets B.V., Heerlen (NL)

(72) Inventors: Elkin AMAYA, Getzersdorf (AT); Estel CANET-MARTINEZ, Getzersdorf (AT); Joshua Thomas CLAYPOOL, Getzersdorf (AT); John Michael GEREMIA, Getzersdorf (AT); Daniel Beat JOSS, Getzersdorf (AT); Mohammad-Ali NEJAD-EBRAHIMI, Watertown, MA (US); Estefania PEREZ CALVO, Getzersdorf (AT); Wen REN, Getzersdorf (AT); Jerome SCHMEISSER, Getzersdorf (AT); Ghislain SCHYNS, Getzersdorf (AT); Clement SESTER, Getzersdorf (AT)

(21) Appl. No.: 18/839,554

(22) PCT Filed: Feb. 23, 2023
(86) PCT No.: PCT/EP2023/054492

§ 371 Date: Aug. 19, 2024

Related U.S. Application Data
(60) Provisional application No. 63/312,971, filed on Feb. 23, 2022.

(51) Int. Cl. A23K 20/163; A23K 10/18; A23K 20/189; A23K 50/30; A23K 50/75

(52) U.S. Cl. CPC A23K 20/163; A23K 10/18; A23K 20/189; A23K 50/30; A23K 50/75

(57) ABSTRACT

The present invention relates to a method for modulating the level of one or more secondary metabolite(s), and a use for modulating the level of one or more secondary metabolite(s) in the gastrointestinal tract of an animal.

The present invention relates to a method for modulating the level of one or more secondary metabolite(s), and a use for modulating the level of one or more secondary metabolite(s) in the gastrointestinal tract of an animal.

Secondary metabolites are molecules produced by an organism, which molecules do not primarily serve a vital purpose—in contrast to molecules of the primary metabolism. Instead, secondary metabolites rather exert their function in interactions of the producing organism with its environment. For instance, some plant secondary metabolites have antifungal activity to fight off pathogens, while other secondary metabolites are produced to attract insects. Some well known secondary metabolites are caffeine, taxol, nicotine, artemisinin, morphine, botulinum toxin etc. Notably, many secondary metabolites may serve several purposes. For instance, caffeine is believed to be produced by plants as a pesticide, while humans consume caffeine to increase vigilance. Other secondary metabolites produced by one organism may act as a neurotransmitter in another organism.

Neurotransmitters are signaling molecules which allow communication among cells of an organism. Receptors for neurotransmitters may be present e.g. on muscle cells or neurons. Gamma-aminobutyric acid (GABA; CAS-No. 56-12-2; also referred to e.g. 5-aminobutanoic acid) is known to be a main inhibitory neurotransmitter in the brain and the spinal cord. GABA slows or blocks specific nerve signals in the brain, thereby modulating levels of anxiety, fear and/or stress as perceivable by humans and animals. Accordingly, imbalanced GABA activity in the body can lead to associated disorders, such as anxiety disorders. Such disorders associated with suboptimal GABA levels in the body further cause secondary effects which are of particular relevance in animal husbandry such as decreased animal performance.

Tryptophan (sometimes abbreviated as Tryp; in amino acid three-letter-code: Trp or TRP; in amino acid one-letter-code: W) is an amino acid and a central molecule in the biosynthesis pathway(s) involving further important secondary metabolites, such as tryptamine, kynurenine, serotonine, and melatonin. In humans, partitioning of the kynurenergic pathway and serotonergic pathway is reported to stand at 90%: 10% of the tryptophan pool. Pathways of tryptophan metabolism in animals are described e.g. in Liu et al., 2020, Trends in Endocrinology and Metabolism 31:818-833, in particular in FIG. 1 therein.

Despite the far-reaching influence of such secondary metabolites on human and animal wellbeing, convenient means for modulating the levels of secondary metabolites in the body remain to be identified. Notably, controlling the right level of a certain molecule in the body is considered challenging. For instance, direct external supply via food supplements comprising GABA yielded conflicting results with limited efficacy so far. Also, the half-life of GABA in the body is comparatively short (e.g. less than 17 min in mice), thus further reducing the efficacy of direct GABA administration.

In view of the prior art as outlined above, it is an objective of the present invention to provide means and methods for improving animal welfare (e.g. by reduction of anxiety, fear and/or stress) and/or improvement of animal performance, e.g. increase in average body weight gain and/or reduction in feed conversion ratio (FCR).

Surprisingly, this objective is achieved by providing a method for modulating the level of at least one secondary metabolite (e.g. neutrotransmitter) in the body of an animal (e.g. increasing the level of gamma-aminobutyric acid (GABA) in the gastrointestinal tract of the animal; and/or increasing the ratio of kynurenine:tryptophan in the body of the animal; and/or increasing the ratio of peripheral serotonin:tryptophan in the digestive system of the animal; and/or increasing the ratio of melatonin:tryptophan in the digestive system of the animal; and/or decreasing the ratio of tryptamine:tryptophan in the digestive system of the animal), the method comprising one or more of the following steps: i) administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; ii) administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or iii) administering an enzyme composition to the animal, wherein the enzyme composition comprises a polypeptide comprising any one of the amino acid sequences of SEQ ID NOs: 1-493, or a functionally equivalent variant of said polypeptide. Preferably, the enzyme composition comprises a polypeptide having glutamate decarboxylase activity, i.e. the polypeptide comprises any one of the amino acid sequences of SEQ ID NOs: 1-317, more preferably any one of the amino acid sequences of SEQ ID NOs: 1-15, or a functionally equivalent variant of said polypeptide.

A person having skill in the art is aware that a certain chemical reaction may sometimes be catalyzed by structurally diverse catalysts. For instance, the decarboxylation of L-glutamate to form GABA may be effected by various polypeptides having different structures, e.g. any one of the amino acid sequences of SEQ ID NOs 1-15. Therefore, a functionally equivalent variant as referred to herein is to be understood as a polypeptide capable of catalyzing the same reaction as the polypeptide to which it is compared (herein: a polypeptide comprising or consisting of any one of the amino acid sequences of SEQ ID NOs: 1-493). However, it is also known that polypeptides having a high degree of similarity or identity to one another share a common enzymatic activity. Therefore, in one embodiment of the invention, the functionally equivalent variant comprised in the enzyme composition of the invention has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493. The degree of sequence identity in percent can be calculated by a suitable algorithm known to a person having skill in the art, e.g. via the Clustal Omega tool at the EMBL-EBI homepage (https://www.ebi.ac.uk/Tools/msa/clustalo/; Sievers et al. 2011. Mol. Syst. Biol. 7:539), or by using the Needleman-Wunsch algorithm e.g. at the National Center for Biotechnology Information homepage (https://blast.ncbi.nlm.nih.gov/Blast.cgi?PAGE_TYPE=BlastSearch&PROG_DEF=blastn&BLAST_PROG_DEF=blastn&BLAST_SPEC=GlobalAIn&LINK_LOC=BlastHomeLink).

SEQ ID NO: 1 (UniProt ID: W0EW32) is a glutamate decarboxylase from Barnesiella viscericola. SEQ ID NO: 2 (UniProt ID: Q5LI66) is a glutamate decarboxylase from Bacteroides fragilis. SEQ ID NO: 3 (UniProt ID: A0A174GPJ1) is a glutamate decarboxylase from Bacteroides finegoldii. SEQ ID NO: 4 (UniProt ID: I9SDM2) is a glutamate decarboxylase from Bacteroides nordii. SEQ ID NO: 5 (UniProt ID: E5CHD8) is a glutamate decarboxylase from Bacteroides sp. D2. SEQ ID NO: 6 (UniProt ID: B3CAZ7) is a glutamate decarboxylase from Bacteroides intestinalis. SEQ ID NO: 7 (UniProt ID: A0A174U6X0) is a glutamate decarboxylase from Bacteroides caccae. SEQ ID NO: 8 (UniProt ID: A0A1M5C9N0) is a glutamate decarboxylase from Bacteroides faecichinchillae. SEQ ID NO: 9 (UniProt ID: Q8A4M9) is a glutamate decarboxylase from Bacteroides thetaiotaomicron. SEQ ID NO: 10 (UniProt ID: F3PY53) is a glutamate decarboxylase from Bacteroides fluxus. SEQ ID NO: 11 (UniProt ID: A0A 139KCP3) is a glutamate decarboxylase from Bacteroides uniformis. SEQ ID NO: 12 (UniProt ID: A0A 120A232) is a glutamate decarboxylase from Bacteroides stercoris. SEQ ID NO: 13 (UniProt ID: A6LA77) is a glutamate decarboxylase from Parabacteroides distasonis. SEQ ID NO: 14 (UniProt ID: A0A374WD41) is a glutamate decarboxylase from Bacteroides sp. OM05-12. SEQ ID NO: 15 (UniProt ID: A0A0P0GIF1) is a glutamate decarboxylase from Bacteroides cellulosilyticus. Therein, SEQ ID NO: 1 is 81.46, 82.29, 82.22, 82.29, 80.83, 81.88, 81.46, 81.46, 81.04, 81.59, 82.01, 83.92, 82.49, 81.04 percent identical to SEQ ID NO: 2, 3, 4, 5, 6, 7, 8, 9, 10, 11, 12, 13, 14, 15, respectively. SEQ ID NO: 2 is 81.46, 94.58, 93.31, 93.75, 94.58, 93.33, 93.54, 93.75, 93.12, 92.68, 92.68, 88.31, 87.97, 94.58 percent identical to SEQ ID NO: 1, 3, 4, 5, 6, 7, 8, 9, 10, 11, 12, 13, 14, 15, respectively. SEQ ID NO: 3 is 82.29, 94.58, 91.84, 96.25, 95.62, 95.62, 94.79, 95.62, 95.42, 96.03, 95.82, 88.94, 87.76, 95.62 percent identical to SEQ ID NO: 1, 2, 4, 5, 6, 7, 8, 9, 10, 11, 12, 13, 14, 15, respectively. SEQ ID NO: 4 is 82.22, 93.31, 91.84, 93.1, 92.68, 92.05, 92.68, 91.63, 91.63, 92.89, 93.1, 88.91, 89.03, 93.1 percent identical to SEQ ID NO: 1, 2, 3, 5, 6, 7, 8, 9, 10, 11, 12, 13, 14, 15, respectively. SEQ ID NO: 5 is 82.29, 93.75, 96.25, 93.1, 94.38, 97.08, 93.96, 96.67, 95.00, 95.19, 95.61, 87.06, 87.97, 94.38 percent identical to SEQ ID NO: 1, 2, 3, 4, 6, 7, 8, 9, 10, 11, 12, 13, 14, 15, respectively. SEQ ID NO: 6 is 80.83, 94.58, 95.62, 92.68, 94.38, 94.58, 94.17, 95.00, 95.00, 96.44, 95.82, 88.1, 87.55, 99.58 percent identical to SEQ ID NO: 1, 2, 3, 4, 5, 7, 8, 9, 10, 11, 12, 13, 14, 15, respectively. SEQ ID NO: 7 is 81.88, 93.33, 95.62, 92.05, 97.08, 94.58, 93.96, 96.88, 96.04, 96.03, 96.03, 87.27, 87.76, 94.58 percent identical to SEQ ID NO: 1, 2, 3, 4, 5, 6, 8, 9, 10, 11, 12, 13, 14, 15, respectively. SEQ ID NO: 8 is 81.46, 93.54, 94.79, 92.68, 93.96, 94.17, 93.96, 94.8, 95.01, 94.56, 94.98, 87.27, 86.29, 94.17 percent identical to SEQ ID NO: 1, 2, 3, 4, 5, 6, 7, 9, 10, 11, 12, 13, 14, 15, respectively. SEQ ID NO: 9 is 81.46, 93.75, 95.62, 91.63, 96.67, 95.00, 96.88, 94.8, 96.67, 94.98, 95.4, 86.22, 87.55, 95.00 percent identical to SEQ ID NO: 1, 2, 3, 4, 5, 6, 7, 8, 10, 11, 12, 13, 14, 15, respectively. SEQ ID NO: 10 is 81.04, 93.12, 95.42, 91.63, 95.00, 95.00, 96.04, 95.01, 96.67, 96.03, 96.44, 87.27, 87.76, 95.00 percent identical to SEQ ID NO: 1, 2, 3, 4, 5, 6, 7, 8, 9, 11, 12, 13, 14, 15, respectively. SEQ ID NO: 11 is 81.59, 92.68, 96.03, 92.89, 95.19, 96.44, 96.03, 94.56, 94.98, 96.03, 97.91, 88.28, 87.55, 96.44 percent identical to SEQ ID NO: 1, 2, 3, 4, 5, 6, 7, 8, 9, 10, 12, 13, 14, 15, respectively. SEQ ID NO: 12 is 82.01, 92.68, 95.82, 93.1, 95.61, 95.82, 96.03, 94.98, 95.4, 96.44, 97.91, 88.49, 87.55, 95.82 percent identical to SEQ ID NO: 1, 2, 3, 4, 5, 6, 7, 8, 9, 10, 11, 13, 14, 15, respectively. SEQ ID NO: 13 is 83.92, 88.31, 88.94, 88.91, 87.06, 88.1, 87.27, 87.27, 86.22, 87.27, 88.28, 88.49, 88.82, 88.52 percent identical to SEQ ID NO: 1, 2, 3, 4, 5, 6, 7, 8, 9, 10, 11, 12, 14, 15, respectively. SEQ ID NO: 14 is 82.49, 87.97, 87.76, 89.03, 87.97, 87.55, 87.76, 86.29, 87.55, 87.76, 87.55, 87.55, 88.82, 87.97 percent identical to SEQ ID NO: 1, 2, 3, 4, 5, 6, 7, 8, 9, 10, 11, 12, 13, 15, respectively. SEQ ID NO: 15 is 81.04, 94.58, 95.62, 93.1, 94.38, 99.58, 94.58, 94.17, 95.00, 95.00, 96.44, 95.82, 88.52, 87.97 percent identical to SEQ ID NO: 1, 2, 3, 4, 5, 6, 7, 8, 9, 10, 11, 12, 13, 14, respectively. SEQ ID NOs 15-317 are further examples of glutamate decarboxylases. SEQ ID NOs 318-323 are examples of gamma-glutamylputrescine synthetases. SEQ ID NOs 324-384 are examples of NADP/NAD-dependent aldehyde dehydrogenases. SEQ ID NOs 385-386 are examples of gamma-glutamyl-gamma-aminobutyrate hydrolases. SEQ ID NOs 387-418 are examples of putrescine aminotransferases. SEQ ID NOs 419-493 are examples of gamma-aminobutyraldehyde dehydrogenases.

Due to the role of GABA in modulating anxiety, fear and/or stress, an internal increase in GABA levels as achievable by the present invention can be found to lead to reduced anxiety, fear and/or stress and thus to improved animal welfare. Further, levels of tryptophan, tryptamine, kynurenine, tryptamine, serotonine, and melatonin can be found to be modulated upon application of the means, methods, uses of the invention, thus allowing further improvements in animal welfare and performance.

Tryptamine is a trace amine neuromodulator (Gao et al. 2018 Front Cell Infect Microbiol 8:13), similar to the cathecholamine neurotransmitters. Trace amines have effects both on the central nervous system (and are therefore involved in the so-called gut-brain axis), but also in the gut lumen where they act on enterocytes. As a trace amine, tryptamine is believed to act as agonist on trace amine-associated receptor TAAR1, involved in energy metabolism and immunomodulation, thereby mediating a host-nutrition-microbiota dialog (Gainetdinov et al. 2018 Pharmacol Rev 70 (3): 549-620).

It has been also demonstrated that tryptamine produced by a gut microbe was able to accelerate the whole gut transit (Bhattarai et al, 2018), therefore being able to influence nutrient absorption. Reduction of tryptamine is therefore favorable for increased animal performance.

Kynurenine (KYN) is known as a neuromodulator of stress. Birkl et al. (2020, Front Vet Sci 6:209) described the KYN/TRP ratio specifically to relate to feather pecking, and to social disturbance in laying hens in general. It was found that a lower KYN/TRP ratio is linked to higher social disturbance profile. Without wishing to be bound to theory, the inventors hypothesize that an increase of the kynurenine/tryptophan ratio is responsible for reducing social disturbance behavior, thus improving animal welfare.

Serotonin within the central nervous system cannot cross the blood/brain barrier, but tryptophan can. Therefore, higher tryptophan in the gut means more tryptophan will cross the blood/brain barrier, where it is then available to be transformed into central serotonin. Serotonin is the precursor of melatonin. An increase in serotonin level will thus cause an increase in melatonin level. It is known that melatonin and its precursor serotonin can impact the production of insulin and glucagon. An increase in the melatonin concentration can enhance the level of insulin and glucagon in animal body. It is also known that increased levels of insulin and glucagon enhance the synthesis of fat. Both insulin and melatonin are involved in regulating circadian rhythm (Wang et al., 2020 PeerJ 8: e9638). Change in the light cycle affect the level of insulin and melatonin produced by an animal. The changed level of insulin and melatonin in the body of the animal in turn regulates the animal's physiological response to the light cycle change. Poultry production in general, and broiler rearing process is now going to long light time, as much as 23 hours a day. This illumination regimen strongly impacts production performance such as faster fat gain but is detrimental to animal welfare.

In another aspect, the invention relates to a use (e.g. a non-medical use) of an oligosaccharide preparation; and/or of a probiotic composition; and/or of an enzyme composition for modulating the level of at least one secondary metabolite (e.g. neutrotransmitter) in the body of an animal (e.g. increasing the level of gamma-aminobutyric acid (GABA) in the gastrointestinal tract of the animal; and/or increasing the ratio of kynurenine: tryptophan in the body of the animal; and/or increasing the ratio of peripheral serotonin: tryptophan in the digestive system of the animal; and/or increasing the ratio of melatonin: tryptophan in the digestive system of the animal; and/or decreasing the ratio of tryptamine: tryptophan in the digestive system of the animal), wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and wherein the enzyme composition comprises a polypeptide comprising any one of the amino acid sequences of SEQ ID NOs: 1-493, or a functionally equivalent variant of said polypeptide.

In another aspect, the invention relates to a use (e.g. a non-medical use) of an oligosaccharide preparation; and/or of a probiotic composition; and/or of an enzyme composition to improve animal performance (e.g. improve average body weight gain and/or reduce feed conversion ratio); and/or to improve animal welfare (e.g. reduce anxiety, stress and/or fear disorders), wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and wherein the enzyme composition comprises a polypeptide comprising any one of the amino acid sequences of SEQ ID NOs: 1-493, or a functionally equivalent variant of said polypeptide.

In some embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein i) the relative abundance of 4-aminobutyrate transaminase activity in the gastrointestinal tract of the animal is reduced; and/or wherein ii) the relative abundance of glutamate decarboxylase activity in the gastrointestinal tract of the animal is increased; and/or wherein iii) the relative abundance of gamma-glutamylputrescine synthetase (e.g. PuuA) activity in the gastrointestinal tract of the animal is increased; and/or wherein iv) the relative abundance of NADP/NAD-dependent aldehyde dehydrogenase (e.g. PuuC) activity in the gastrointestinal tract of the animal is increased; and/or wherein v) the relative abundance of gamma-glutamyl-gamma-aminobutyrate hydrolase (e.g. PuuD) activity in the gastrointestinal tract of the animal is increased; and/or wherein vi) the relative abundance of putrescine aminotransferase activity in the gastrointestinal tract of the animal is increased; and/or wherein vii) the relative abundance of gamma-aminobutyraldehyde dehydrogenase activity in the gastrointestinal tract of the animal is increased. Merely for clarification, 4-aminobutyrate transaminase activity is described by the Enzyme Commission number (EC number; Webb, E. C. 1992. Enzyme nomenclature 1992: recommendations of the Nomenclature Committee of the International Union of Biochemistry and Molecular Biology on the nomenclature and classification of enzymes. Academic Press. ISBN 978-0-12-227164-9) 2.6.1.19. Glutamate decarboxylase activity (i.e. glutamate decarboxylase enzymes) is described by EC 4.1.1.15. Gamma-glutamylputrescine synthetase activity is described by EC 6.3.1.11. NADP/NAD-dependent aldehyde dehydrogenase activities can be described by EC 1.2.1.5. Gamma-glutamyl-gamma-aminobutyrate hydrolase is described by EC 3.5.1.94. Putrescine aminotransferase is described by EC 2.6.1.82. Gamma-aminobutyraldehyde dehydrogenase is described by EC 1.2.1.19. The EC numbers disclosed herein can be found e.g. on the BRENDA database on enzyme functional data (www.brenda-enzymes.org; Chang A. et al. 2021. Nucleic Acids Res. 49: D498-D508. Schomburg I. et al. 2000. Gene Funct. Dis. 3-4:109-118). While 4-aminobutyrate transaminase has been found to reduce GABA concentration, glutamate decarboxylase, gamma-glutamylputrescine synthetase, NADP/NAD-dependent aldehyde dehydrogenase, gamma-glutamyl-gamma-aminobutyrate hydrolase, putrescine aminotransferase as well as gamma-aminobutyraldehyde dehydrogenase have been found to direct biosynthesis pathways towards formation of GABA. By performing the method for modulating the level of at least one secondary metabolite (e.g. neutrotransmitter) in the body of an animal referred to herein; or the use for modulating the level of at least one secondary metabolite (e.g. neutrotransmitter) in the body of an animal referred to herein; or the use for improving animal performance and/or animal welfare referred to herein, wherein i) the relative abundance of 4-aminobutyrate transaminase activity in the gastrointestinal tract of the animal is reduced; and/or wherein ii) the relative abundance of glutamate decarboxylase activity in the gastrointestinal tract of the animal is increased; and/or wherein iii) the relative abundance of gamma-glutamylputrescine synthetase (e.g. PuuA) activity in the gastrointestinal tract of the animal is increased; and/or wherein iv) the relative abundance of NADP/NAD-dependent aldehyde dehydrogenase (e.g. PuuC) activity in the gastrointestinal tract of the animal is increased; and/or wherein v) the relative abundance of gamma-glutamyl-gamma-aminobutyrate hydrolase (e.g. PuuD) activity in the gastrointestinal tract of the animal is increased; and/or wherein vi) the relative abundance of putrescine aminotransferase activity in the gastrointestinal tract of the animal is increased; and/or wherein vii) the relative abundance of gamma-aminobutyraldehyde dehydrogenase activity in the gastrointestinal tract of the animal is increased, it was found possible to achieve an increase in GABA concentration in the animal without external supply with GABA and without the drawbacks associated with such external supply, such as the short half-life of GABA when provided by external supply.

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein the level of GABA in the brain of the animal is increased at least 5% (e.g. at least 10%, 15%, 20%, 25%, 30%, 35%; or 10-35%) compared to the level of GABA in the brain of an animal not treated by the method, or not administered the oligosaccharide preparation, the probiotic composition and/or the enzyme composition.

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein the animal is selected from the group consisting of pet animals, dog, cat, canary, guinea pig, hamster, rabbit, mouse, rat, deer, boar, zoo animals, horse, donkey, poultry, swine, ruminant, chicken, cow, sheep, goat, pig, piglet, turkey, aquaculture, fish, shrimp, prawn, crayfish, crab, oyster, mussel, clam, trout, tilapia, salmon, carp, catfish, tuna, preferably, the animal is selected from the group consisting of poultry such as chicken, swine, and ruminant such as cow.

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein the oligosaccharide preparation is comprised in a nutritional composition administered to an animal at an inclusion rate of at least 50 ppm (e.g. at least 50, 70, 100, 150, 200, 300, 400, 500 ppm).

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein n (of the oligosaccharide preparation) is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

In further embodiments, the invention relates to the method for modulating the level of at least one secondary metabolite (e.g. neutrotransmitter) in the body of an animal referred to herein; or to the use for modulating the level of at least one secondary metabolite (e.g. neutrotransmitter) in the body of an animal referred to herein; or to the use for improving animal performance and/or animal welfare referred to herein, wherein the relative abundance of oligosaccharides in each of the n fractions of the oligosaccharide preparation decreases monotonically with its degree of polymerization. By chosing the oligosaccharide preparation to have one or more of the features described herein, a particular improvement of both, animal performance as well as welfare can be achieved. For instance, such an oligosaccharide preparation inter alia surprisingly increases the relative abundance of glutamate decarboxylase activity in the gastrointestinal tract of the animal, and further the level of GABA in the animal body.

In another aspect, the invention relates to an oligosaccharide preparation; and/or a probiotic composition; and/or an enzyme composition for use in treatment, amelioration and/or prophylaxis of one or more disorders associated with an imbalanced level of GABA and/or an imbalanced ratio of kynurenine: tryptophan, serotonin: tryptophan, melatonin: tryptophan, and/or tryptamine: tryptophan, wherein the one or more disorders are selected from the group of anxiety, stress, fear disorders, systemic inflammation, and local inflammation; wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry; wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and wherein the enzyme composition comprises a polypeptide comprising any one of the amino acid sequences of SEQ ID NOs: 1-493, or a functionally equivalent variant of said polypeptide.

It has been observed that the health of the host animal, animal welfare and/or animal performance are improved in four aspects by the means, methods and uses of the invention. First, welfare of the group of production animals is improved. It is a common problem for monogastric animals such as chicken and ducks raised in a confined space to develop social disturbance behaviors such as feather pecking or tail biting. Disturbance behaviors like this cause poor welfare of the production animal and thus has been a persisting problem for animal farmers. The means, methods and uses according to the invention help to improve the welfare of animals.

Second, the health of the host animal can be improved by way of decreasing systemic inflammation of the animal. Systemic inflammation is the result of release of pro-inflammatory cytokines from immune-related cells and the chronic activation of the innate immune system. It contributes to the development of chronical disease conditions in animals. The method according to the invention helps to reduce systemic inflammation of the animal.

Third, the health of the host animal can be improved by way of decreasing local inflammation of the animal. Local inflammation occurs within the area affected by the harmful stimulus. Acute local inflammation develops within minutes or hours following a harmful stimulus, has a short duration, and primarily involves the innate immune system. The method according to the invention helps to reduce local inflammation of the animal.

Fourth, the health of the host animal can be improved by way of reducing the light regimen/duration into the daily circadian rhythm of the animal (Soliman and Hassan 2019 Veterinary World 12 (7): 1052-1059). The circadian rhythms associated with light have important effects on the growth of production animals. In the production animal farming business, one way for increasing the growth rate and meat production is by prolongation of the illumination. In some extreme cases, the illumination on poultry is extended to 23 hours a day, leaving the poultry under darkness for only one hour a day. Although such a method may increase productivity, it has negative impacts on the health as well as the welfare of the animal. It has been observed that the melatonin level of chicken under the 23 hours light and 1 hour darkness period treatment was lowered to less than half of the amount of melatonin of the chicken which are under the 16 hours light and 8 hours darkness period treatment. The means, methods and uses according to the present invention help to increase the amount of melatonin and its precursor serotonin and thus restore the level of melatonin in animals which are subjected to prolonged illumination. Since artificially prolonged photoperiod leads to abnormal behavior such as aggressive interactions (tail biting, feather pecking, mobility/motility issues etc.) in poultry, restoring of melatonin level in such animals helps to improve the welfare of the animals. The inventors of the present application have discovered that by compensating melatonin production by applying the means, methods and uses according to the invention, a stronger serotonergic flux is going into more melatonin and thus a reduction of the illumination regimen and a better animal welfare can be achieved.

The means and methods and uses of the present invention are applicable to production animals in general and may be provided to any suitable animal. In some embodiments, the animal is monogastric. It is generally understood that a monogastric animal has a single-chambered stomach. In other embodiments, the animal is a ruminant. It is generally understood that a ruminant has a multi-chambered stomach. In some embodiments, the animal is a ruminant in the pre-ruminant phase. Examples of such ruminants in the pre-ruminant phase include nursery calves.

In some embodiments, the animal is a poultry (e.g. chicken, turkey), seafood (e.g. shrimp), sheep, cow, cattle, buffalo, bison, pig (e.g. nursery pig, grower/finisher pig), cat, dog, rabbit, goat, guinea pig, donkey, camel, horse, pigeon, ferret, gerbil, hamster, mouse, rat, bird, or human.

In some embodiments, the animal is livestock. In some embodiments, the animal is a companion animal. In some embodiments, the animal is poultry. Examples of poultry include chicken, duck, turkey, goose, quail, or Cornish game hen. In one variation, the animal is a chicken. In some embodiments, the poultry is a layer hen, a broiler chicken, or a turkey.

In other embodiments, the animal is a mammal, including, for example, a cow, a pig, a goat, a sheep, a deer, a bison, a rabbit, an alpaca, a llama, a mule, a horse, a reindeer, a water buffalo, a yak, a guinea pig, a rat, a mouse, an alpaca, a dog, or a cat. In one variation, the animal is a cow. In another variation, the animal is a pig. In another variation, the animal is a sow.

The invention is further characterized by the following items:
Item 1: Method for modulating, in particular increasing the level of gamma-aminobutyric acid (GABA) in the gastrointestinal tract of an animal, the method comprising one or more of the following steps:
   administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   administering an enzyme composition to the animal, wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 2: Method for increasing the ratio of kynurenine: tryptophan in the body of an animal, the method comprising one or more of the following steps:
   administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   administering an enzyme composition to the animal, wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 3: Method for increasing the ratio of peripheral serotonin: tryptophan in the digestive system of an animal, the method comprising one or more of the following steps:
   administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   administering an enzyme composition to the animal, wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 4: Method for increasing the ratio of melatonin: tryptophan in the digestive system of an animal, the method comprising one or more of the following steps:
   administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   administering an enzyme composition to the animal, wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 5: Method for decreasing the ratio of tryptamine: tryptophan in the digestive system of an animal, the method comprising one or more of the following steps:
   administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   administering an enzyme composition to the animal, wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 6: Use (e.g. non-medical use) of an oligosaccharide preparation; and/or of a probiotic composition; and/or of an enzyme composition
   for modulating, in particular increasing the level of gamma-aminobutyric acid (GABA) in the gastrointestinal tract of an animal,
   wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 7: Use (e.g. non-medical use) of an oligosaccharide preparation; and/or of a probiotic composition; and/or of an enzyme composition
   for increasing the ratio of kynurenine: tryptophan in the body of an animal,
   wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 8: Use (e.g. non-medical use) an oligosaccharide preparation; and/or of a probiotic composition; and/or of an enzyme composition
   for increasing the ratio of peripheral serotonin: tryptophan in the digestive system of an animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 9: Use (e.g. non-medical use) of an oligosaccharide preparation; and/or of a probiotic composition; and/or of an enzyme composition
   for increasing the ratio of melatonin: tryptophan in the digestive system of an animal,
   wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 10: Use (e.g. non-medical use) of an oligosaccharide preparation; and/or of a probiotic composition; and/or of an enzyme composition
   for decreasing the ratio of tryptamine: tryptophan in the digestive system of an animal,
   wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 11: Use (e.g. non-medical use) of an oligosaccharide preparation; and/or of a probiotic composition; and/or of an enzyme composition to improve animal performance (e.g. improve average body weight gain and/or reduce feed conversion ratio); and/or to improve animal welfare (e.g. reduce anxiety, stress and/or fear disorders),
   wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 12: The method according to any one of items 1-5, wherein the relative abundance of 4-aminobutyrate transaminase activity in the gastrointestinal tract of the animal is reduced; wherein the relative abundance of glutamate decarboxylase activity in the gastrointestinal tract of the animal is increased; wherein the relative abundance of gamma-glutamylputrescine synthetase (e.g. PuuA) activity in the gastrointestinal tract of the animal is increased; wherein the relative abundance of NADP/NAD-dependent aldehyde dehydrogenase (e.g. PuuC) activity in the gastrointestinal tract of the animal is increased; wherein the relative abundance of gamma-glutamyl-gamma-aminobutyrate hydrolase (e.g. PuuD) activity in the gastrointestinal tract of the animal is increased; wherein the relative abundance of putrescine aminotransferase activity in the gastrointestinal tract of the animal is increased; and/or wherein the relative abundance of gamma-aminobutyraldehyde dehydrogenase activity in the gastrointestinal tract of the animal is increased.
Item 13: The method according to any one of items 1-5 or 12, wherein the level of GABA in the brain of the animal is increased at least 5% (e.g. at least 10%, 15%, 20%, 25%, 30%, 35%; or 10-35%) compared to the level of GABA in the brain of an animal not treated by the method.
Item 14: The method according to any one of items 1-5 or 12-13, wherein the animal is selected from the group consisting of pet animals, dog, cat, canary, guinea pig, hamster, rabbit, mouse, rat, deer, boar, zoo animals, horse, donkey, poultry, swine, ruminant, chicken, cow, sheep, goat, pig, piglet, turkey, aquaculture, fish, shrimp, prawn, crayfish, crab, oyster, mussel, clam, trout, tilapia, salmon, carp, catfish, tuna, preferably, the animal is selected from the group consisting of poultry such as chicken, swine, ruminant such as cow.
Item 15: The method according to any one of items 1-5 or 12-14, wherein the oligosaccharide preparation is comprised in a nutritional composition administered to an animal at an inclusion rate of at least 50 ppm (e.g. at least 50, 70, 100, 150, 200, 300, 400, 500 ppm).
Item 16: The method according to any one of items 1-5 or 12-15, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 17: The method according to any one of items 1-5 or 12-16, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 18: The method according to any one of items 1-5 or 12-17, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 19: The method according to any one of items 1-5 or 12-18, wherein the relative abundance of oligosaccharides in each of the n fractions of the oligosaccharide preparation decreases monotonically with its degree of polymerization.
Item 20: The use according to any one of items 6-11, wherein the relative abundance of 4-aminobutyrate transaminase activity in the gastrointestinal tract of the animal is reduced; wherein the relative abundance of glutamate decarboxylase activity in the gastrointestinal tract of the animal is increased; wherein the relative abundance of gamma-glutamylputrescine synthetase (e.g. PuuA) activity in the gastrointestinal tract of the animal is increased; wherein the relative abundance of NADP/NAD-dependent aldehyde dehydrogenase (e.g. PuuC) activity in the gastrointestinal tract of the animal is increased; wherein the relative abundance of gamma-glutamyl-gamma-aminobutyrate hydrolase (e.g. PuuD) activity in the gastrointestinal tract of the animal is increased; wherein the relative abundance of putrescine aminotransferase activity in the gastrointestinal tract of the animal is increased; and/or wherein the relative abundance of gamma-aminobutyraldehyde dehydrogenase activity in the gastrointestinal tract of the animal is increased.
Item 21: The use according to any one of items 6-11 or 20, wherein the level of GABA in the brain of the animal is increased at least 5% (e.g. at least 10%, 15%, 20%, 25%, 30%, 35%; or 10-35%) compared to the level of GABA in the brain of an animal not treated by the method.
Item 22: The use according to any one of items 6-11 or 20-21, wherein the animal is selected from the group consisting of pet animals, dog, cat, canary, guinea pig, hamster, rabbit, mouse, rat, deer, boar, zoo animals, horse, donkey, poultry, swine, ruminant, chicken, cow, sheep, goat, pig, piglet, turkey, aquaculture, fish, shrimp, prawn, crayfish, crab, oyster, mussel, clam, trout, tilapia, salmon, carp, catfish, tuna, preferably, the animal is selected from the group consisting of poultry such as chicken, swine, ruminant such as cow.
Item 23: The use according to any one of items 6-11 or 20-22, wherein the oligosaccharide preparation is comprised in a nutritional composition administered to an animal at an inclusion rate of at least 50 ppm (e.g. at least 50, 70, 100, 150, 200, 300, 400, 500 ppm).
Item 24: The use according to any one of items 6-11 or 20-23, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 25: The use according to any one of items 6-11 or 20-24, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 26: The use according to any one of items 6-11 or 20-25, wherein wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 27: The use according to any one of items 6-11 or 20-26, wherein the relative abundance of oligosaccharides in each of the n fractions of the oligosaccharide preparation decreases monotonically with its degree of polymerization.
Item 28: An oligosaccharide preparation; and/or a probiotic composition; and/or an enzyme composition for use in treatment, amelioration and/or prophylaxis of one or more disorders associated with an imbalanced level of GABA and/or an imbalanced ratio of kynurenine: tryptophan, serotonin: tryptophan, melatonin: tryptophan, and/or tryptamine: tryptophan,
   wherein the one or more disorders are selected from the group of anxiety, stress, fear disorders, systemic inflammation, and local inflammation;
   wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
   wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and
   wherein the enzyme composition comprises a polypeptide comprising or consisting of any one of the amino acid sequences of SEQ ID NOs: 1-493, or a functionally equivalent variant of said polypeptide, preferably wherein the functionally equivalent variant comprised in the enzyme composition has at least 70% (e.g. at least 75.0%, 80.0%, 80.8%, 81%, 82%, 83%, 84%, 85%, 86%, 87%, 88%, 89%, 90%, 91%, 92%, 93%, 94%, 95%, 96%, 97%, 98%, 99%, 99.0%, 99.5%, etc.) sequence identity to any one of the amino acid sequences of SEQ ID NOs: 1-493.
Item 29: The method according to any one of items 1-5 or 12-19; and/or the use according to any one of items 6-11 or 20-27, wherein the microbiome in the gastrointestinal tract is modulated.

The oligosaccharide preparation referred to herein (e.g. in the items) can be further characterized by any one of the following features, or by more than one (e.g. two, three, four, five, ten or more, or even all) of the following features (e.g. by feature a); a) and b); a) and b) and c); a)-d); a)-e), and so forth until a)-aaa); but also by individual features such as f); ww); or specific combinations thereof such as d) and z) and xx); f)-bb) and qq)-yy) etc.):
a) the oligosaccharide preparation comprises at least n fractions of oligosaccharides each having a distinct degree of polymerization selected from 1 to n (DP1 to DPn fractions), wherein n is an integer greater than or equal to 2, wherein each fraction comprises from at least about 0.5% to about 90% (e.g. from 1% to 90%; from about 0.5% to about 15%) of anhydro-subunit containing oligosaccharides by relative abundance as determined by mass spectrometry;
b) the oligosaccharide preparation is comprised in a nutritional composition administered to an individual (e.g. an animal);
c) the oligosaccharide preparation is comprised in the nutritional composition at an inclusion rate of at least 50 ppm (e.g. at least 50, 70, 100, 150, 200, 300, 400, 500 ppm);
d) n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100;
e) at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance;
f) at least one fraction of the oligosaccharide preparation comprises less than 10%, 9%, 8%, 7%, 6%, 5%, 4%, 3%, or 2% anhydro-subunit containing oligosaccharides by relative abundance;
g) each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance;
h) the oligosaccharide preparation has a weight average molecular weight from about 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol;
i) the oligosaccharide preparation has a weight average molecular weight from about 2000 to 2800 g/mol, 2100 to 2700 g/mol, 2200 to 2600 g/mol, 2300 to 2500 g/mol, or 2320 to 2420 g/mol;
j) the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol;
k) the relative abundance of oligosaccharides in each of the n fractions of the oligosaccharide preparation decreases monotonically with its degree of polymerization;
l) the relative abundance of oligosaccharides in at least 5, 10, 20, or 30 DP fractions of the oligosaccharide preparation decreases monotonically with its degree of polymerization;
m) at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance;
n) the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance;
o) each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance;
p) at least one fraction of the oligosaccharide preparation comprises less than 10%, 9%, 8%, 7%, 6%, 5%, 4%, 3%, or 2% anhydro-subunit containing oligosaccharides by relative abundance;
q) the oligosaccharide preparation comprises less than 10%, 9%, 8%, 7%, 6%, 5%, 4%, 3%, or 2% anhydro-subunit containing oligosaccharides by relative abundance;
r) each fraction of the oligosaccharide preparation comprises less than 10%, 9%, 8%, 7%, 6%, 5%, 4%, 3%, or 2% anhydro-subunit containing oligosaccharides by relative abundance;
s) at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance;
t) the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance;
u) each fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance;
v) at least one fraction of the oligosaccharide preparation comprises greater than 20%, 21%, 22%, 23%, 24%, or 25% anhydro-subunit containing oligosaccharides by relative abundance;
w) the oligosaccharide preparation comprises greater than 20%, 21%, 22%, 23%, 24%, or 25% anhydro-subunit containing oligosaccharides by relative abundance;
x) each fraction of the oligosaccharide preparation comprises greater than 20%, 21%, 22%, 23%, 24%, or 25% anhydro-subunit containing oligosaccharides by relative abundance;
y) more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit;
z) the oligosaccharide preparation has a DP1 fraction content from 1 to 40% by relative abundance;
aa) the oligosaccharide preparation has a DP2 fraction content from 1 to 35% by relative abundance;
bb) the oligosaccharide preparation has a DP3 fraction content from 1 to 30% by relative abundance;
cc) the oligosaccharide preparation has a DP4 fraction content from 0.1 to 20% by relative abundance;
dd) the oligosaccharide preparation has a DP5 fraction content from 0.1 to 15% by relative abundance;
hh) the ratio of DP2 fraction to DP1 fraction of the oligosaccharide preparation is 0.02-0.40 by relative abundance;
ii) the ratio of DP3 fraction to DP2 fraction of the oligosaccharide preparation is 0.01-0.30 by relative abundance;
jj) the aggregate content of DP1 and DP2 fractions in the oligosaccharide preparation is less than 50, 30, or 10% by relative abundance;
kk) the oligosaccharide preparation comprises at least 103, 104, 105, 106 or 109 different oligosaccharide species;
ll) two or more independent oligosaccharides of the oligosaccharide preparation comprise different anhydro-subunits;
mm) the oligosaccharide preparation comprises one or more anhydro-subunits that are products of reversible thermal dehydration of monosaccharides;
nn) the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits;
oo) the oligosaccharide preparation comprises one or more anhydro-glucose, anhydro-galactose, anhydro-mannose, or anhydro-fructose subunits;
pp) the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits;
qq) a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation;
rr) the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation;
ss) the ratio of 1,6-anhydro-β-D-glucofuranose to 1,6-anhydro-β-D-glucopyranose is about 2:1 in the oligosaccharide preparation;
tt) the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation;
uu) the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation;
vv) the ratio of 1,6-anhydro-β-D-glucofuranose to 1,6-anhydro-β-D-glucopyranose is about 2:1 in each fraction of the oligosaccharide preparation;
ww) at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose;
xx) the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol;
yy) the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol;
zz) the distribution of the degree of polymerization is determined and/or detected by MALDI-MS, GC-MS, LC-MS, SEC, HPLC and/or combination(s) thereof (e.g. MALDI-MS and SEC); and/or
aaa) the degree of polymerization of the oligosaccharide preparation may be determined based on its molecular weight and molecular weight distribution.

It is also considered that the oligosaccharide preparation may be provided in the form of a powderous formulation comprising at least 20% (w/w) of the oligosaccharide preparation as referred to herein; at least 25% (wt/wt) of a silica-based adsorbate (e.g. diatomaceous earth, amorphous precipitated silica) having an average particle size D of less than or equal to 3000 μm (e.g. 100-500, 200-500, 200-300 μm); and optionally 0-25% (wt/wt) of water and/or an auxiliary substance; wherein the % are based on the total weight of the powderous formulation.

For instance, such a powderous formulation may comprise 30-70% (wt/wt) of the oligosaccharide preparation as referred to herein; 30-70% (wt/wt) of a silica based adsorbate (e.g. having an average particle size of at least 50 μm); and 0-21% (wt/wt) of water; wherein the % are based on the total weight of the powderous formulation. In some embodiments the oligosaccharide preparation is formulated as described in any one of Examples 22-26 and 33 of WO 2020/097458.

Descriptions and methods of manufacturing oligosaccharide preparations according to the invention are described in WO 2020/097458, WO 2016/007778, in particular in the Examples described therein, in particular in any one of Examples 1-7, 16-18 of WO 2020/097458 A1, in the methods described in paragraph [317], and/or in any one of Examples 73-77, 80-89, 97-99, 101-110 of WO 2016/007778 A1. In particular, oligosaccharide preparations according to the invention are characterized by the step of heating an aqueous composition comprising one or more feed sugars and a catalyst to a temperature and for a time sufficient to induce polymerization.

Merely for the sake of clarity, the term “oligosaccharide preparation” may refer to a preparation that comprises one or more oligosaccharides.

As used herein, an “oligosaccharide” or “oligomer” may refer to a monosaccharide or a compound containing two or more monosaccharide subunits linked by glycosidic bonds. An “oligosaccharide” may also refer to an anhydro-monosaccharide or a compound containing two or more monosaccharide subunits, where at least one monosaccharide unit is replaced by an anhydro-subunit. An “oligosaccharide” may be optionally functionalized. As used herein, the term “oligosaccharide” encompasses all species of the oligosaccharide, wherein each of the monosaccharide subunit in the oligosaccharide is independently and optionally functionalized and/or replaced with its corresponding anhydro-monosaccharide subunit.

An “anhydro-subunit” may be a product of reversible thermal dehydration of a monosaccharide (or monosaccharide subunit) or a sugar caramelization product. For example, an “anhydro-subunit” may be an anhydro-monosaccharide such as anhydro-glucose. As another example, an “anhydro-subunit” may be linked with one or more regular or anhydro-monosaccharide subunits via glycosidic linkage.

An oligosaccharide may be characterized to contain two or more monosaccharide subunits linked by glycosidic bonds. In this regard, a “gluco-oligosaccharide” may refer to a glucose or a compound containing two or more glucose monosaccharide subunits linked by glycosidic bonds. A “gluco-oligosaccharide” may also refer to an anhydro-glucose or a compound containing two or more glucose monosaccharide subunits linked by glycosidic bonds, wherein at least one monosaccharide subunit is replaced with an anhydro-glucose subunit. Similarly, a “galacto-oligosaccharide” may refer to a galactose or a compound containing two or more galactose monosaccharide subunits linked by glycosidic bonds. A “galacto-oligosaccharide” may also refer to an anhydro-galactose or a compound containing two or more galactose monosaccharide subunits linked by glycosidic bonds, wherein at least one monosaccharide subunit is replaced with an anhydro-galactose subunit. Analogously, a gluco-galactose-oligosaccharide may be a gluco-oligosaccharide, a galacto-oligosaccharide, or a compound containing one or more glucose monosaccharide subunits and one or more galactose monosaccharide subunits linked by glycosidic bonds, wherein at least one of the monosaccharide subunits is replaced with its respective anhydro-monosaccharide subunit. A gluco-galacto-xylo-oligosaccharide may refer to a compound produced by the condensation reaction of glucose, galactose, and xylose. An oligosaccharide preparation comprising gluco-galacto-xylo-oligosaccharides may comprise gluco-galactose-oligosaccharides, gluco-xylo-oligosaccharides, galacto-xylo-oligosaccharides, and compounds containing one or more glucose monosaccharide subunits, one or more xylose monosaccharide subunits, and one or more galactose monosaccharide subunits linked by glycosidic bonds.

As used herein, the term “monosaccharide unit” and “monosaccharide subunit” may be used interchangeably, unless suggested otherwise. A “monosaccharide subunit” may refer to a monosaccharide monomer in an oligosaccharide. For an oligosaccharide having a degree of polymerization of 1, the oligosaccharide may be referred to as a monosaccharide subunit or monosaccharide. For an oligosaccharide having a degree of polymerization higher than 1, its monosaccharide subunits are linked via glycosidic bonds.

As used herein, the term “regular monosaccharide” may refer to a monosaccharide that does not contain an anhydro-subunit. The term “regular disaccharide” may refer to a disaccharide that does not contain an anhydro-subunit. Accordingly, the term “regular subunit” may refer to a subunit that is not an anhydro-subunit.

The term “relative abundance” or “abundance,” as used herein, may refer to the abundance of a species in terms of how common or rare the species exists. For example, a DP1 fraction comprising 10% anhydro-subunit containing oligosaccharides by relative abundance may refer to a plurality of DP1 oligosaccharides, wherein 10%, by number, of the DP1 oligosaccharides are anhydro-monosaccharides.

Degree of Polymerization (DP) Distribution: A distribution of the degree of polymerization of the oligosaccharide preparation may be determined by any suitable analytical method and instrumentation, including but not limited to end group method, osmotic pressure (osmometry), ultracentrifugation, viscosity measurements, light scattering method, size exclusion chromatography (SEC), SEC-MALLS, field flow fractionation (FFF), asymmetric flow field flow fractionation (A4F), high-performance liquid chromatography (HPLC), and mass spectrometry (MS). For example, the distribution of the degree of polymerization may be determined and/or detected by mass spectrometry, such as MALDI-MS, LC-MS, or GC-MS. For another example, the distribution of the degree of polymerization may be determined and/or detected by SEC, such as gel permeation chromatography (GPC). As yet another example, the distribution of the degree of polymerization may be determined and/or detected by HPLC, FFF, or A4F. In another example, the degree of polymerization of the oligosaccharide preparation may be determined based on its molecular weight and molecular weight distribution (for a more detailed description see WO 2020/097458).

Anhydro-subunit Level: In some embodiments, each of the n fractions of oligosaccharides of the oligosaccharide preparation as described herein independently comprises an anhydro-subunit level. For instance, in some embodiments, the DP1 fraction comprises 10% anhydro-subunit containing oligosaccharides by relative abundance, and the DP2 fraction comprises 15% anhydro-subunit containing oligosaccharides by relative abundance. For another example, in some embodiments, DP1, DP2, and DP3 fraction each comprises 5%, 10%, and 2% anhydro-subunit containing oligosaccharides by relative abundance, respectively. In other embodiments, two or more fractions of oligosaccharides may comprise similar level of anhydro-subunit containing oligosaccharides. For example, in some embodiments, the DP1 and DP3 fraction each comprises about 5% anhydro-subunit containing oligosaccharides by relative abundance.

The level of anhydro-subunits may be determined by any suitable analytical methods, such as nuclear magnetic resonance (NMR) spectroscopy, mass spectrometry, HPLC, FFF, A4F, or any combination thereof. In some embodiments, the level of anhydro-subunits is determined, at least in part, by mass spectrometry such as MALDI-MS. In some embodiments, the level of anhydro-subunits may be determined, at least in part, by NMR. In some embodiments, the level of anhydro-subunits may be determined, at least in part, by HPLC. For example, in some embodiments, the level of anhydro-subunits may be determined by MALDI-MS, as illustrated in more detail in WO 2020/097458.

Glycosidic Linkages: In some embodiments, the oligosaccharide preparation described herein comprise a variety of glycosidic linkages. The type and distribution of the glycosidic linkages may depend on the source and manufacturing method of the oligosaccharide preparation. In some embodiments, the type and distribution of various glycosidic linkages may be determined and/or detected by any suitable methods known in the art such as NMR. For example, in some embodiments, the glycosidic linkages are determined and/or detected by proton NMR, carbon NMR, 2D NMR such as 2D JRES, HSQC, HMBC, DOSY, COSY, ECOSY, TOCSY, NOESY, or ROESY, or any combination thereof. In some embodiments, the glycosidic linkages are determined and/or detected, at least in part, by proton NMR. In some embodiments, the glycosidic linkages are determined and/or detected, at least in part, by carbon NMR. In some embodiments, the glycosidic linkages are determined and/or detected, at least in part, by 2D HSQC NMR.

In some embodiments, an oligosaccharide preparation may comprise one or more α-(1,2) glycosidic linkages, α-(1,3) glycosidic linkages, α-(1,4) glycosidic linkages, α-(1,6) glycosidic linkages, 3-(1,2) glycosidic linkages, β-(1,3) glycosidic linkages, β-(1,4) glycosidic linkages, β-(1,6) glycosidic linkages, α(1,1)α glycosidic linkages, α(1,1) β glycosidic linkages, β(1,1)β glycosidic linkages, or any combination thereof.

In some embodiments, the oligosaccharide preparations have a glycosidic bond type distribution of about from 0 to 60 mol %, 5 to 55 mol %, 5 to 50 mol %, 5 to 45 mol %, 5 to 40 mol %, 5 to 35 mol %, 5 to 30 mol %, 5 to 25 mol %, 10 to 60 mol %, 10 to 55 mol %, 10 to 50 mol %, 10 to 45 mol %, 10 to 40 mol %, 10 to 35 mol %, 15 to 60 mol %, 15 to 55 mol %, 15 to 50 mol %, 15 to 45 mol %, 15 to 40 mol %, 15 to 35 mol %, 20 to 60 mol %, 20 to 55 mol %, 20 to 50 mol %, 20 to 45 mol %, 20 to 40 mol %, 20 to 35 mol %, 25 to 60 mol %, 25 to 55 mol %, 25 to 50 mol %, 25 to 45 mol %, 25 to 40 mol %, or 25 to 35 mol % of a-(1,6) glycosidic linkages.

Molecular Weight: The molecular weight and molecular weight distribution of the oligosaccharide preparation may be determined by any suitable analytical means and instrumentation, such as end group method, osmotic pressure (osmometry), ultracentrifugation, viscosity measurements, light scattering method, SEC, SEC-MALLS, FFF, A4F, HPLC, and mass spectrometry. In some embodiments, the molecular weight and molecular weight distribution are determined by mass spectrometry, such as MALDI-MS, LC-MS, or GC-MS. In some embodiments, the molecular weight and molecular weight distribution are determined by size exclusion chromatography (SEC), such as gel permeation chromatography (GPC). In other embodiments, the molecular weight and molecular weight distribution are determined by HPLC. In some embodiments, the molecular weight and molecular weight distribution are determined by MALDI-MS.

In some embodiments, the weight average molecular weight of the oligosaccharide preparation is about from 100 to 10000 g/mol, 200 to 8000 g/mol, 300 to 5000 g/mol, 500 to 5000 g/mol, 700 to 5000 g/mol, 900 to 5000 g/mol, 1100 to 5000 g/mol, 1300 to 5000 g/mol, 1500 to 5000 g/mol, 1700 to 5000 g/mol, 300 to 4500 g/mol, 500 to 4500 g/mol, 700 to 4500 g/mol, 900 to 4500 g/mol, 1100 to 4500 g/mol, 1300 to 4500 g/mol, 1500 to 4500 g/mol, 1700 to 4500 g/mol, 1900 to 4500 g/mol, 300 to 4000 g/mol, 500 to 4000 g/mol, 700 to 4000 g/mol, 900 to 4000 g/mol, 1100 to 4000 g/mol, 1300 to 4000 g/mol, 1500 to 4000 g/mol, 1700 to 4000 g/mol, 1900 to 4000 g/mol, 300 to 3000 g/mol, 500 to 3000 g/mol, 700 to 3000 g/mol, 900 to 3000 g/mol, 1100 to 3000 g/mol, 1300 to 3000 g/mol, 1500 to 3000 g/mol, 1700 to 3000 g/mol, 1900 to 3000 g/mol, 2100 to 3000 g/mol, 300 to 2500 g/mol, 500 to 2500 g/mol, 700 to 2500 g/mol, 900 to 2500 g/mol, 1100 to 2500 g/mol, 1300 to 2500 g/mol, 1500 to 2500 g/mol, 1700 to 2500 g/mol, 1900 to 2500 g/mol, 2100 to 2500 g/mol, 300 to 1500 g/mol, 500 to 1500 g/mol, 700 to 1500 g/mol, 900 to 1500 g/mol, 1100 to 1500 g/mol, 1300 to 1500 g/mol, 2000-2800 g/mol, 2100-2700 g/mol, 2200-2600 g/mol, 2300-2500 g/mol, or 2320-2420 g/mol. In some embodiments, the weight average molecular weight of the oligosaccharide preparation is about from 2000 to 2800 g/mol, 2100 to 2700 g/mol, 2200 to 2600 g/mol, 2300 to 2500 g/mol, or 2320 to 2420 g/mol.

Types of Oligosaccharides: In some embodiments, the species of oligosaccharides present in an oligosaccharide preparation referred to herein may depend on the type of the one or more feed sugars. For example, in some embodiments, the oligosaccharide preparations comprise a gluco-oligosaccharide when the feed sugars comprise glucose. For example, in some embodiments, the oligosaccharide preparations comprise a galacto-oligosaccharide when the feed sugars comprise galactose. For another example, in some embodiments, the oligosaccharide preparations comprise gluco-galacto-oligosaccharides when the feed sugars comprise galactose and glucose.

In some embodiments, the oligosaccharide preparations comprise one or more species of monosaccharide subunits. In some embodiments, the oligosaccharide preparation may comprise oligosaccharides with 1, 2, 3, 4, 5, 6, 7, 8, 9, 10, 11, 12, 13, 14, 15, 16, 17, 18, 19, 20, or more different species of monosaccharides subunits.

Method of Manufacturing Oligosaccharide Preparations: The Method of manufacturing an oligosaccharide preparation according to the invention is described in detail in WO 2020/097458 comprising heating an aqueous composition comprising one or more feed sugars and a catalyst to a temperature and for a time sufficient to induce polymerization, wherein the catalyst is selected from the group consisting of: (+)-camphor-10-sulfonic acid; 2-pyridinesulfonic acid; 3-pyridinesulfonic acid; 8-hydroxy-5-quinolinesulfonic acid hydrate; α-hydroxy-2-pyridinemethanesulfonic acid; (β)-camphor-10-sulfonic acid; butylphosphonic acid; diphenylphosphinic acid; hexylphosphonic acid; methylphosphonic acid; phenylphosphinic acid; phenylphosphonic acid; tert-butylphosphonic acid; SS)-VAPOL hydrogenphosphate; 6-quinolinesulfonic acid, 3-(1-pyridinio)-1-propanesulfonate; 2-(2-pyridinyl) ethanesulfonic acid; 3-(2-pyridyl)-5,6-diphenyl-1,2,4-triazine-p,p′-disulfonic acid monosodium salt hydrate; 1,1′-binaphthyl-2,2′-diyl-hydrogenphosphate; bis(4-methoxyphenyl)phosphinic acid; phenyl(3,5-xylyl)phosphinic acid; L-cysteic acid monohydrate; poly(styrene sulfonic acid-co-divinylbenzene); lysine; Ethanedisulfonic acid; Ethanesulfonic acid; Isethionic acid; Homocysteic acid; HEPBS (N-(2-Hydroxyethyl)piperazine-N′-(4-butanesulfonic acid)); HEPES (4-(2-hydroxyethyl)-1-piperazineethanesulfonic acid); 2-Hydroxy-3-morpholinopropanesulfonic acid; 2-(N-morpholino)ethanesulfonic acid; Methanesulfonic acid; Methaniazide; Naphthalene-1-sulfonic acid; Naphthalene-2-sulfonic acid; Perfluorobutanesulfonic acid; 6-sulfoquinovose; Triflic acid; 2-aminoethanesulfonic acid; Benzoic acid; Chloroacetic acid; Trifluoroacetic acid; Caproic acid; Enanthic acid; Caprylic acid; Pelargonic acid; Lauric acid; Pamitic acid; Stearic acid; Arachidic acid; Aspartic acid; Glutamic acid; Serine; Threonine; Glutamine; Cysteine; Glycine; Proline; Alanine; Valine; Isoleucine; Leucine; Methionine; Phenylalanine; Tyrosine; Tryptophan.

In some embodiments, the polymerization of the feed sugars is achieved by a step-growth polymerization. In some embodiments, the polymerization of the feed sugars is achieved by polycondensation.

Feed Sugar: The one or more feed sugars used in the methods of manufacturing oligosaccharide preparations described herein may comprise one or more types of sugars. In some embodiments, the one or more feed sugars comprise monosaccharides, disaccharides, trisaccharides, tetrasaccharides, or any mixtures thereof.

In some embodiments, the one or more feed sugars comprise glucose. In some embodiments, the one or more feed sugars comprise glucose and galactose. In some embodiments, the one or more feed sugars comprise glucose, xylose, and galactose. In some embodiments, the one or more feed sugars comprise glucose and mannose. In some embodiments, the one or more feed sugars comprise glucose and fructose. In some embodiments, the one or more feed sugars comprise glucose, fructose, and galactose. In some embodiments, the one or more feed sugars comprise glucose, galactose, and mannose.

As used herein, the singular forms “a,” “and,” and “the” include plural referents unless the context clearly dictates otherwise. Thus, for example, reference to “an agent” includes a plurality of such agents, and reference to “the oligosaccharide” includes reference to one or more oligosaccharides (or to a plurality of oligosaccharides) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

In the following, the present invention is further described by non-limiting examples.

### EXAMPLES

The present invention as disclosed herein is not limited to specific embodiments, figures, methodology, examples, protocols etc. described herein but solely defined by the claims.

### Example 1

A feeding trial was performed to study the effects of oligosaccharide preparations on the performance and welfare of birds in husbandry. The test period began on Trial Day 0 (day of hatch of chicks), when chicks began being fed a commercial-type feed in pelleted form (further crumbled for Starter feeds), and ended on Trial Day 42. Each experimental unit contained 40 male broilers (Hubbard-Cobb) randomly assigned into 21 replicates per group for a total number of 840 animals per treatment on study. Broiler chicks were randomly assigned to treatments of Trial Day 0 (or on day of hatch) and were not replaced during the course of the trial. The chicks were observed daily for signs of unusual grow-out patterns or health problems. Body weights, feed consumption and feed conversion were measured on Trial Days 0, 10, 24, and 42. Cecal content samples, ileal tissue samples, and blood plasma samples were collected from 1 bird per pen at 24 and 42 days of age. For the vaccination program, all birds received Marek's vaccine, as well as being sprayed with vaccine against coccidiosis and for Newcastle bronchitis. No feed grade antibiotics were administered during the course of the study. All birds were grown on new litter. Feed and water were provided ad libitum throughout the conduct of the study.

The commercial-simulated test model employed in this study used broiler chicks (Gallus gallus domesticus) reared under a normal poultry industry Starter diet (0-10 days of age), Grower diet (11-24 days of age) and Finisher diet (25-42 days of age) at a floor space requirement of a minimum of 0.85 ft² per bird, reared in floor pens with new litter. Ration formulations were conducted via computer-generated linear regression program that simulates formulations conducted during practical poultry production techniques. Treatments were tested in male broilers. Broilers were continuously fed their experimental diets from time of placement on Trial Day 0 (day of hatch) to 42 days of age. All diets contained 1000 FYT/kg of phytase (RONOZYME® HiPhos).

| | | Test Material |
|---|---|---|
| | | Inclusion |
| Treatment | Description | (ppm) |
| Control | high/poor quality protein | 0 |
| Test | Control + oligosaccharide | 500 |
| | preparation | |

Broiler chicks were weighed and randomly placed into each pen on day of hatch (Trial Day 0) and fed their respective diets. Each pen had sufficient floor density, feeder and waterer space for each grow-out area for chickens up to 42 days of age. Following 42 days of grow-out, broilers were weighed, feed consumption determined, and feed conversion ratio (feed consumed/body weight) calculated and adjusted for mortality.

Oligosaccharide preparations comprise at least n fractions of oligosaccharides each having a distinct degree of polymerization selected from 1 to n (DP1 to DPn fractions), wherein n is an integer greater than 3; wherein each of a DP1 and DP2 fraction independently comprises from about 0.5% to about 15% of anhydro-subunit containing oligosaccharides by relative abundance as determined by mass spectrometry. Oligosaccharide preparations were produced as disclosed in WO 2020/097458, WO 2016/007778, in particular as described in the Examples section therein.

Test material description: Test material was provided in either liquid or powder form, and mixed into the treatment feeds. The treatment feeds were then pelleted (and further crumbled for the Starter feeds) and placed into the pens according to the pen design for this study. Treatments were fed continuously from Trial Days 0-42. Test material treatments (comprising oligosaccharide preparation according to the invention) were compared to a Control treatment (not comprising said oligosaccharide preparation).

Experimental design: A total of 8,000 male broiler chicks (a sufficient number to ensure availability of at least 7,560 healthy male chicks for the conduct of the study) were obtained from a commercial hatchery on Trial Day 0 (same as hatch date). These were immediately transported to the feeding trial facility under temperature-controlled conditions to assure bird comfort. After arrival at the facility, broilers were immediately randomized. There were 40 healthy/viable male broilers per pen with 21 pens per test group for a total of 840 broilers per treatment group. Broilers were fed their respective treatment feed ad libitum from day of hatch (Trial Day 0) to 42 days of age.

Detailed broiler chick description: Animal care practices conformed to the Guide for the Care and Use of Agricultural Animals in Agricultural Research and Teaching (FASS, 2010, 3rd Edition). Commercial broilers (Hubbard-Cobb) were obtained at hatch (Trial Day 0) from a commercial hatchery. Broilers were evaluated upon receipt for signs of disease or other complications that may have affected the outcome of the study. Following examination, broilers were weighed. Broilers were allocated to each pen and to treatment groups using a randomized block design. Weight distribution across the treatment groups were assessed prior to feeding by comparing the individual test group standard deviations of the mean against that of the control group. Differences between control and test groups were within one standard deviation, and as such, weight distribution across treatment groups were considered acceptable for this study.

Broiler chicks (on day of hatch, called Day 0) were collected in the early morning and were randomly assigned to each experimental pen within 12 hours of hatch. Weak birds were removed and humanely sacrificed. Birds were not replaced during the study.

Housing and daily observations: Each experimental test unit of broiler mixed-sex chicken pens were housed in separated pens, located in a room containing forced air heaters with a cross-house ventilation system. Broilers were placed in a 5 ft×10 ft pen floor area with a minimum of 0.85 ft² per bird (without feeder and waterer space) provided. At least two nipple drinkers per pen (via well water) were provided.

Feeders were employed for the grow-out period and checked daily to ensure that all birds had access to feed at all times.

The light program employed made use of incandescent lighting for approximately 23 hours of continuous light and 1 hour of darkness per day for Days 0-7, and for approximately 20 hours of continuous light and 4 hours of darkness per day for the remainder of the study.

Birds were observed daily for overall health, behavior and/or evidence of toxicity, and environmental conditions. Temperature in the test facility was checked daily. Drinking water and feed were confirmed to be provided ad libitum.

No type of medication (other than test material) was administered during the entire feeding period. Mortalities were collected daily and body weights recorded on all broilers found dead or moribund.

Data and observations: Live performance body weights and feed intakes were collected on Days 0, 10, 24, and 42 during the growing period. Weight gain, feed intake, feed: gain ratio (feed efficiency) were calculated for 0-42 days of age and other age periods between hatch and market weights. Differences between broilers fed control and test groups were statistically evaluated at P<0.05 in a typical ANOVA analysis of variance test model, employing Treatment x Replicate RCB (Randomized Complete Block). Control group was considered to be the following: Treatment 1, with no added test materials.

At the end of the study, all carcasses of necropsied broilers and all birds remaining at the end of the study, after being humanely euthanized, were disposed of according to local regulations via on-farm composting techniques.

Diet Preparation: A basal ration for each phase was formulated to meet or exceed minimum nutrient requirements of a typical commercial broiler diet using formulations employed by qualified nutritionist with training in poultry feed formulations, and formulated rations met or exceeded NRC Nutrient Requirements for Poultry as a guideline (9th edition, 1994). Feed formulations were furnished by a veterinarian, conducted by a regression analysis program commonly used for Least-Cost Feed Formulation in the poultry industry. Test materials were then mixed into the basal ration.

Dietary protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production as normal animal feed compositions. Mixing equipment was flushed with ground corn prior to each diet preparation. All diets were prepared using a paddle mixer. The mixer was cleaned between each diet using compressed air and vacuum, mixing equipment was flushed with ground corn between each treatment group, and flush material was retained for disposal.

Diet and water administration: Diets were fed in three feed phases: Starter diet (0-10 days of age), Grower diet (11-24 days of age) and Finisher diet (25-42 days of age). All diets were offered ad libitum, without restriction. Fresh well water (from the research facility deep well) was provided ad libitum.

Feed Formulation Parameters:

| | Starter | Grower | Finisher |
|---|---|---|---|
| Ingredients, % | (d0-11) | (d11-24) | (d24-42) |
| Corn | 58.353 | 65.383 | 69.413 |
| SBM, 47.9% CP | 29.992 | 20.404 | 16.879 |
| Anim & veg fat | 0.05 | 0.05 | 0.050 |
| DL-methionine | 0.515 | 0.4.77 | 0.420 |
| Salt | 0.342 | 0.332 | 0.332 |
| L-Lysine-HCL | 0.367 | 0.439 | 0.400 |
| Limestone | 1.794 | 1.537 | 1.470 |
| Dical Phos | 1.079 | 0.869 | 0.739 |
| Choline CL-70% | 0.044 | 0.080 | 0.091 |
| TM & Vit Premix | 0.075 | 0.075 | 0.075 |
| Wheat midds | 2.377 | 0.344 | 0.121 |
| Corn DDGs | 5.0 | 10.00 | 10.00 |
| Ronozyme HiPhos | 0.01 | 0.01 | 0.01 |
| Nutrient | Amount | Amount | Amount |
| Protein, % | 20.3 | 17.5 | 16.0 |
| Ca, % | 0.920 | 0.770 | 0.720 |
| Tphos, % | 0.656 | 0.578 | 0.533 |
| Avphos, % | 0.350 | 0.300 | 0.270 |
| Sodium, % | 0.160 | 0.160 | 0.160 |
| ME kcal/kg | 2900 | 3040 | 3084 |
| Av Lys, % | 1.33 | 1.140 | 1.010 |
| Av M + C, % | 0.99 | 0.880 | 0.790 |
| Choline, g/kg | 1.60 | 1.60 | 1.60 |

Measurement and sampling schedule: On days 0, 10, 24 and 42: Performance; BWG, FI and FCR (corrected and uncorrected for mortality) Per pen basis. On days 24 and 42: Cecal samples (1 bird/pen), 21 reps/trt; Ileal tissue (1 bird/pen), 21 reps/trt; Plasma (1 bird/pen),21 reps/trt. On day 0 (before bird placement) and day 42: Litter samples (one composite sample per pen), 21 reps/trt (3 in front, 3 in the middle, and 3 in the back).

Results: The test period began on Trial Day 0 (day of hatch of chicks), and chicks were fed a commercial-type feed in pelleted form (crumbles on Days 0-10) until the end of the study. Each treatment contained 21 replicates per treatment randomly assigned and containing 40 male broilers per replicate. Chicks were randomly assigned to treatments on Trial Day 0 (or day of hatch). At 42 days of age, live performance (growth weight gain, mortality and feed conversion) and other criteria were determined.

With respect to daily observations, each pen was closely monitored each day to determine overall health, bird behavior and/or evidence of toxicity, and environmental conditions.

Temperature was checked within the growing area employed for this study daily. Temperature program employed for this study was maintaining temperatures of approximately 86+/−5° F. for the first seven (7) days, decreasing approximately 1° F. per day thereafter until a target of approximately 70+/−5° F. was reached, which was maintained throughout the study.

For the entire grow-out period (Days 0-42), body weight gain showed significant improvement over the Control group when broilers were fed diets containing the oligosaccharide preparation Feed conversion for Trial Days 0-42 followed a similar patter as final body weights. Mortality was considered average for this breed in all groups throughout the growing period to 42 days of age without significant differences. Normal poultry industry mortality is typically <4.5% when birds are grown on litter bedding floors.

Observed data on average body weight, feed conversion ratio (corrected for mortality), mortality in %, and average body weight gain is shown in the subsequent table. Statistical evaluation is for each observation is shown in the respective row below.

| Data | Control | Test |
|---|---|---|
| Average body weight in kg, Day 10 | 0.3623 | 0.3684 |
| Statistical evaluation¹ | ^{a} | ^{a} |
| Feed conversion ratio (corrected), Day 0-10 | 1.167 | 1.152 |
| Statistical evaluation¹ | ^{a} | ^{b} |
| Mortality in %, Day 0-10 | 1.19 | 1.31 |
| Statistical evaluation¹ | ^{a} | ^{a} |
| Average body weight gain in kg, Day 0-10 | 0.3184 | 0.3248 |
| Statistical evaluation¹ | ^{a} | ^{a} |
| Average body weight in kg, Day 24 | 1.0484 | 1.0756 |
| Statistical evaluation¹ | ^{a} | ^{b} |
| Feed conversion ratio (corrected), Day 0-24 | 1.413 | 1.379 |
| Statistical evaluation¹ | ^{a} | ^{b} |
| Mortality in %, Day 0-24 | 2.262 | 2.857 |
| Statistical evaluation¹ | ^{a} | ^{a} |
| Average body weight gain in kg, Day 0-24 | 1.0045 | 1.0319 |
| Statistical evaluation¹ | ^{a} | ^{b} |
| Average body weight in kg, Day 42 | 2.5071 | 2.5755 |
| Statistical evaluation¹ | ^{a} | ^{b} |
| Feed conversion ratio (corrected), Day 0-42 | 1.833 | 1.783 |
| Statistical evaluation¹ | ^{a} | ^{b} |
| Mortality in %, Day 0-42 | 3.297 | 4.762 |
| Statistical evaluation¹ | ^{a} | ^{a} |
| Average body weight gain in kg, Day 0-42 | 2.4632 | 2.5318 |
| Statistical evaluation¹ | ^{a} | ^{b} |
| Feed conversion ratio (corrected), Day 10-24 | 1.529 | 1.486 |
| Statistical evaluation¹ | ^{a} | ^{b} |
| Mortality in %, Day 10-24 | 1.09 | 1.579 |
| Statistical evaluation¹ | ^{a} | ^{a} |
| Average body weight gain in kg, Day 10-24 | 0.6861 | 0.7071 |
| Statistical evaluation¹ | ^{a} | ^{b} |
| Feed conversion ratio (corrected), Day 24-42 | 2.136 | 2.075 |
| Statistical evaluation¹ | ^{a} | ^{b} |
| Mortality in %, Day 24-42 | 1.003 | 1.886 |
| Statistical evaluation¹ | ^{a} | ^{a} |
| Average body weight gain in kg, Day 24-42 | 1.4587 | 1.4999 |
| Statistical evaluation¹ | ^{a} | ^{b} |

¹Means within a row without a common superscript are significantly different (P < 0.05) as determined by Least Significant Difference.

When using shotgun metagenomic sequencing as described above of cecum samples from chickens, nucleid acid sequences from the microbiome encoding gadB activity were searched. Similar to the results obtained from feeding trial 1 described above, genes encoding gadB activity were found relatively more abundant in samples from animals of the test group (i.e. those animals that were fed with a diet comprising the oligosaccharide preparation as described herein) compared to animals of the control group. In other words, animals being fed with the oligosaccharide preparation described herein comprise a higher abundance of the GABA-producing gadB activity, in particular in the gastrointestinal tract, compared to animals not being fed the oligosaccharide preparation. As a consequence, animals being fed the oligosaccharide preparation have a higher GABA availability and are thus less likely to show anxiety-, fear- or stress-related behavior.

Profiling of the composition of microbial communities from metagenomic shotgun sequencing data was performed by using a bioinformatic tool called MetaPhlAn3 (Metagenomic Phylogenetic Analysis; Beghini et al. 2021. eLife 2021; 10: e65088. DOI: 10.7554/eLife.65088). Microbial species present in the samples were analyzed for the presence of sequences encoding gadB by BLAST searches. The species that contained gabB and that were present in the samples were then identified as following: Barnesiella viscericola; Bacteroides uniformis; Bacteroides cellulosilyticus; Bacteroides fragilis. Unexpectedly, these species were found to be more abundant in samples from animals being fed with the oligosaccharide preparation described herein than in samples from animals not being fed with the oligosaccharide preparation. It is hypothesized, that an observed reduction in anxiety-, fear- or stress-related behavior can be attributed to the increased abundance of one or more of these species.

### Example 2

A library of synthetic oligosaccharide preparations was prepared using different combinations of feed sugar(s) and catalysts. Feed sugars were selected from glucose, lactose, galactose, mannose, glucosamine, N-acetylglucosamine, arabinose, xylose, sucrose, and fructose. Catalysts were selected from sulfuric acid, acetic acid, propionic acid, butanoic acid, L-glutamic acid, L-lysine, phosphoric acid, (+)-camphor-10-sulfonic acid, methylphosphonic acid, L-cysteic acid, hydrochloric acid, citric acid, lactic acid, 3-(1-pyridinio)-1-propanesulfonate, 2-pyridinesulfonic acid, 3-pyridinesulfonic acid, α-hydroxy-2-pyridinemethanesulfonic acid, (β)-camphor-10-sulfonic acid, butylphosphonic acid, diphenylphosphinic acid, hexylphosphonic acid, phenylphosphinic acid, phenylphosphonic acid, tert-butylphosphonic acid, 2-(2-pyridinyl)ethanesulfonic acid, 3-(2-pyridyl)-5,6-diphenyl-1,2,4-triazine-p,p′-disulfonic acid monosodium salt hydrate, 1,1′-binaphthyl-2,2′-diyl-hydrogenphosphate, bis(4-methoxyphenyl)phosphinic acid, and phenyl (3,5-xylyl) phosphinic acid.

In principle, 10 g-scale condensation reactions were performed by charging respective masses of powdered sugars (dry solids basis) into 20 mL scintillation vials, melting the contents by heating to 135° C. while stirring, and allowing for oligomerization to occur by adding catalyst and continuing heating to evolve water off of the reaction vessel for up to several hours until adequate conversion was achieved as indicated by increased viscosity, brown or amber color. In detail, the sugars glucose, N-acetylglucosamine, lactose were used at a ratio of 68:2:30 and condensed using (+)-camphor-10-sulfonic acid as catalyst. The sugars glucose, arabinose, N-acetylglucosamine were used at a ratio of 94:5:1 and condensed using (+)-camphor-10-sulfonic acid as catalyst. The sugars glucose, fructose, N-acetylglucosamine were used at a ratio of 92:5:3 and condensed using (+)-camphor-10-sulfonic acid as catalyst. The sugars glucose, sucrose, N-acetylglucosamine were used at a ratio of 94:5:1 and condensed using (+)-camphor-10-sulfonic acid as catalyst. The sugars glucose, sucrose, N-acetylglucosamine were used at a ratio of 92:5:3 and condensed using (+)-camphor-10-sulfonic acid as catalyst. The sugars glucose, N-acetylglucosamine were used at a ratio of 99:1 and condensed using (+)-camphor-10-sulfonic acid as catalyst. The sugars glucose, N-acetylglucosamine were used at a ratio of 98:2 and condensed using (+)-camphor-10-sulfonic acid as catalyst. The sugars glucose, N-acetylglucosamine were used at a ratio of 97:3 and condensed using (+)-camphor-10-sulfonic acid as catalyst. The sugars glucose, fructose, arabinose were used at a ratio of 90:5:5 and condensed using methylphosphonic acid as catalyst. The sugars glucose, sucrose, arabinose were used at a ratio of 90:5:5 and condensed using methylphosphonic acid as catalyst. The sugars glucose, xylose, arabinose were used at a ratio of 90:5:5 and condensed using methylphosphonic acid as catalyst. The sugars glucose, arabinose, lactose were used at a ratio of 85:5:10 and condensed using methylphosphonic acid as catalyst. The sugars glucose, fructose, lactose were used at a ratio of 75:5:20 and condensed using methylphosphonic acid as catalyst. The sugars glucose, N-acetylglucosamine, lactose were used at a ratio of 88:2:10 and condensed using methylphosphonic acid as catalyst. The sugars glucose, sucrose, lactose were used at a ratio of 85:5:10 and condensed using methylphosphonic acid as catalyst. The sugars glucose, xylose, lactose were used at a ratio of 85:5:10 and condensed using methylphosphonic acid as catalyst. The sugars glucose, fructose, mannose were used at a ratio of 85:5:10 and condensed using methylphosphonic acid as catalyst. The sugars glucose, N-acetylglucosamine, mannose were used at a ratio of 94:1:5 and condensed using methylphosphonic acid as catalyst. The sugars glucose, sucrose, mannose were used at a ratio of 85:5:10 and condensed using methylphosphonic acid as catalyst. The sugars glucose, xylose, mannose were used at a ratio of 90:5:5 and condensed using methylphosphonic acid as catalyst. The sugars glucose, arabinose, N-acetylglucosamine were used at a ratio of 94:5:1 and condensed using methylphosphonic acid as catalyst. The sugars glucose, fructose, N-acetylglucosamine were used at a ratio of 94:5:1 and condensed using methylphosphonic acid as catalyst. The sugars glucose, fructose, N-acetylglucosamine were used at a ratio of 92:5:3 and condensed using methylphosphonic acid as catalyst. The sugars glucose, sucrose, N-acetylglucosamine were used at a ratio of 94:5:1 and condensed using methylphosphonic acid as catalyst. The sugars glucose, sucrose, N-acetylglucosamine were used at a ratio of 93:5:2 and condensed using methylphosphonic acid as catalyst. The sugars glucose, sucrose, N-acetylglucosamine were used at a ratio of 92:5:3 and condensed using methylphosphonic acid as catalyst. The sugars glucose, xylose, N-acetylglucosamine were used at a ratio of 93:5:2 and condensed using methylphosphonic acid as catalyst. The sugars glucose, fructose, xylose were used at a ratio of 90:5:5 and condensed using methylphosphonic acid as catalyst. The sugars glucose, sucrose, xylose were used at a ratio of 90:5:5 and condensed using methylphosphonic acid as catalyst. The sugars glucose, arabinose were used at a ratio of 95:5 and condensed using methylphosphonic acid as catalyst. The sugars glucose, lactose were used at a ratio of 90:10 and condensed using methylphosphonic acid as catalyst. The sugars glucose, N-acetylglucosamine were used at a ratio of 99:1 and condensed using methylphosphonic acid as catalyst. The sugars glucose, N-acetylglucosamine were used at a ratio of 98:2 and condensed using methylphosphonic acid as catalyst. The sugars glucose, N-acetylglucosamine were used at a ratio of 97:3 and condensed using methylphosphonic acid as catalyst. The sugars glucose, xylose were used at a ratio of 95:5 and condensed using methylphosphonic acid as catalyst. The sugars glucose, arabinose, N-acetylglucosamine were used at a ratio of 94:5:1 and condensed using phosphoric acid as catalyst. The sugars glucose, fructose, N-acetylglucosamine were used at a ratio of 92:5:3 and condensed using phosphoric acid as catalyst. The sugars glucose, sucrose, N-acetylglucosamine were used at a ratio of 92:5:3 and condensed using phosphoric acid as catalyst.

The amount of catalyst present in the oligosaccharide preparations after production was quantified by anion chromatography. The analysis was performed on a Thermo-Fisher ICS-3000 ion chromatography system equipped with EG-3000 eluent generator, AS autosampler and CD-3000 conductivity detector. Separation was achieved on an AS19 column (4.0 mm×250 mm) protected with an AG19 precolumn (4.0 mm×50 mm). The mobile phase was a KOH gradient (0-55 mM) produced by an eluent generator (EGCII EluGen® cartridge). The chromatographic parameters were: 1.0 mL/min flow rate, 25 μL injection volume, and 30° C. column temperature. Calculations were based on peak areas. Quantitative results were obtained by comparing peak areas with an external standard calibration curve. All catalysts applied could be quantified and were below 0.1%, i.e. 1000 ppm, after the condensation reaction.

Samples of the synthetic oligosaccharide preparations were further analyzed by gel permeation chromatography on an Agilent 1260 Infinity system comprising degasser, binary pump, and thermostat column compartment equipped with an Agilent 1200 refractive index detector. The columns used were a PL aquagel-OH guard column, 7.5×50 mm, 5 M (Agilent Part No: PL1149-1530) and a PL aquagel-OH, 7.5×300 mm, 5 M (Agilent Part No: PL1120-6520), whereas the used solvent was 200 mM aqueous sodium nitrate at a flow-rate of 0.9 mL/min. and a column temperature of 40° C. As standards, both glucose (for the quantification of total sugar content) and pullulan standards (for the size distribution) were used. Oligosaccharide preparations were found to have a DP1 content (DP: degree of polymerization; DP1: degree of polymerization of up to 90%; and to have a DP2+content (oligosaccharides having a degree of polymerization of 2 or more) of at least 10%. For example, the oligosaccharide preparation obtained from glucose, N-acetylglucosamine, and lactose using methylphosphonic acid as catalyst was found to comprise a DP1 content of 69.22 area %, a DP2 content of 24.64 area % and a DP3+content of 6.14 area % as determined via liquid chromatography with evaporative light scattering detector coupled to high-resolution mass spectrometry (LC-ELSD-HRMS). Further, the Mn (number averaged molecular mass), and Mw (weight averaged molecular mass) were both determined to each have at least 300 g/mol and at most 1500 g/mol. All of the prepared oligosaccharide preparations were further found to comprise a content of anhydro-subunit containing oligosaccharides from 0.5% to 15% in each DP fraction.

### Example 3

In order to test for increased GABA production by intestinal pig microbiome, the following assay was performed under anaerobic conditions. To this end, a minimal media solution, a glycan solution (comprising the oligosaccharide preparation to be tested), a feed extract solution, and a swine cecal slurry (comprising intestinal microbiota) were prepared.

The minimal media solution was prepared to contain 900 mg/L Sodium Chloride, 26 mg/L Calcium Chloride dihydrate, 20 mg/L Magnesium chloride hexahydrate, 40 mg/L Ammonium Sulfate, 300 mg/L Potassium Phosphate dibasic, 1500 mg/L Sodium Phosphate dibasic, 5000 mg/L Sodium Bicarbonate, 150 mg/L Histidine, 75 mg/L Glycine, 150 mg/L Tryptophan, 150 mg/L Arginine, 150 mg/L Methionine, 150 mg/L Threonine, 225 mg/L Valine, 225 mg/L Isoleucine, 300 mg/L Leucine, 400 mg/L Cysteine, 450 mg/L Proline, 75 mg/L Phenylalanine, 75 mg/L Tyrosine, 75 mg/L Glutamic Acid, 10 mg/L Manganese Chloride in solution, 4 mg/L Iron Sulfate in solution, 1 mg/L Cobalt Chloride in solution, 1 mg/L Pyridoxine in solution, 1 mg/L Pantothenate in solution, and 0.125 mg/L Biotin in solution.

The glycan solutions were prepared to a concentration of 6 brix of each of the individual oligosaccharide preparations to be tested, diluted with distilled water. To this end, the synthetic oligosaccharide preparations as prepared in Example 2 were used.

The feed extract solution was prepared by mixing ground pig feed (as detailed in Table 4) with 20 mL of distilled water, followed by incubating at 125° C. for 15 min. Thereafter, the mixture was pelleted by centrifugation The supernatant was incubated at 121° C. for 15 min. Thereafter, sterile distilled water was added to a final volume of 20 mL.

TABLE 4
Pig feed composition.

| | Experimental period |
|---|---|
| Ingredients (%) | Finisher |
| Wheat | 28.9 |
| Corn | 40 |
| SBM 48 | 14.3 |
| Rice bran | 8 |
| Premix vitamin-mineral* | 3 |
| Soy oil | 3 |
| L-Lysine | 0.35 |
| L-Methionine | 0.05 |
| NaCl | 0.5 |
| Calcium carbonate | 0.8 |
| Dicalcium phosphate | 1.1 |

Calculated energy & nutrient contents

| | |
|---|---|
| Metabolizable energy (Kcal/kg) | 3132 |
| Fat (%) | 6.9 |
| Crude protein (%) | 15.1 |
| Lysine (%) | 0.94 |
| Methionine (%) | 0.27 |
| Threonine (%) | 0.55 |
| Total P total (%) | 0.58 |
| Total Ca (%) | 0.64 |
| Ratio Ca/P | 0.91 |
| Vit A (IU/kg) | 6571.3 |
| Vit E (mg/kg) | 91.5 |
| Vit D3 (IU/kg) | 2007 |
| Se (mg/kg) | 0.51 |
| Fe (mg/kg) | 175.7 |
| Zn (mg/kg) | 93 |
| Cu (mg/kg) | 20.8 |

*Premix vitamin mineral 3144 provided per kilogram of diet: Vitamin A 6510 I.U.; Vitamin E: 75 mg.; Vitamin K: 2 mg; Vitamin D3: 2007 IU; Vitamin B1: 0.99 mg; Vitamin B2: 5.02 mg; Vitamin B6: 2.01 mg; Vitamin B12: 0.03 mg; Pantothenic acid: 17.5 mg; Folic acid: 0.51 mg; Biotin 0.1 mg; Choline: 100 mg; Mn: 40 mg; Fe: 100 mg; Cu: 15 mg; Zn: 65 mg; I: 2 mg; Se: 0.4 mg.

Swine cecal slurry was prepared by mixing 3.8 g of caecal sample in 25 mL PBS (phosphate-buffered saline: 137 mM sodium chloride, 2.7 mM potassium chloride, 10 mM disodium hydrogenphosphate, 1.8 mM potassium dihydrogen phosphate, pH 7.4). After sedimentation of large particles within this mixture, the non-sedimented supernatant was decanted into a fresh vessel. Thereafter, this supernatant was subjected to centrifugation (500 rpm, 5 min). After centrifugation, the supernatant was recovered and mixed with 50% glycerol to a final glycerol concentration of 15% (v/v), thus forming the swine cecal slurry.

In order to mimic the actual diversity of pig caecal microbiota, three caecal samples comprising different microbiome compositions were tested. As identified metagenomically, the caecal samples differed in terms of diversity as well as in microbial composition and ratio. In detail, in terms of phyla, caecal sample 1 contained 48.0%, 33.3%, 15.4% and 3.3% of Firmicutes, Bacteroidetes, Proteobacteria, and Actinobacteriae, respectively. Caecal sample 2 contained 54.9%, 27.2%, 15.3% and 2.7% of Firmicutes, Bacteroidetes, Proteobacteria, and Actinobacteria, respectively. Caecal sample 3 contained 67.0%, 20.4%, 10.4%, and 2.3% of Firmicutes, Bacteroidetes, Proteobacteria, and Actinobacteriae, respectively. In terms of genera, caecal sample 1 contained 15.1%, 8.5%, 14.6%, 5.2%, 3.8%, 3.6%, 4.2%, 23.4%, 6.2%, 11.1%, and 4.3% of Bacteroides, Blautia, Clostridium, Escherichia, Faecalibacterium, Lachnoclostridium, Phocaeicola, Prevotella, Roseburia, Streptococcus, and Turicibacter, respectively. Caecal sample 2 contained 10.8%. 5.5%. 3.7%. 12.3%. 6.0%. 10.3%. 4.9%. 17.9%. 4.4%. 9.9%. and 14.3% of Bacteroides. Blautia. Campylobacter, Clostridium, Escherichia, Lactobacillus, Limosilactobacillus, Prevotella, Roseburia, Sarcina, and Streptococcus, respectively. Caecal sample 3 contained 6.6%, 5.9%, 4.4%, 1.8%, 2.7%, 41.9%, 18.1%, 2.0%, 10.5%, 4.4%, and 1.8%, of Bacteroides, Blautia, Clostridium, Faecalibacterium, Lachnoclostridium, Lactobacillus, Limosilactobacillus, Phocaeicola, Prevotella, Roseburia, and Ruminococcus, respectively.

Production of GABA by the microbiota was initiated upon mixing 50 μL of the swine cecal slurry with 80 μL of the feed extract solution, 70 μL of the glycan solution and 800 μL of the minimal media solution in a single well of a 96-deep well plate. The glycan solution was varied by individually testing the different oligosaccharide preparations as prepared in Example 2. As controls, a blank control was included, wherein the glycan solution was substituted with minimal media solution; and a glucose control was included, wherein the glycan solution contained 1% (v/v) of glucose instead of an oligosaccharide preparation as referred to herein. The glucose control was included to obtain a basal GABA response from the microbiome as a reference for fold changes calculations. Since glucose is mostly absorbed before actually reaching the caecum in the small intestine, the 1% glucose control appropriately mimicked the in vivo situation. The 96-deep well plates were sealed with a porous, breathable adhesive film, and incubated anaerobically for 24 h at 37° C. Thereafter, the 96-deep well plates were centrifuged for pelleting the microbiota and non-dissolved matter, and the supernatants were used for further analysis, in particular for determination of production of GABA and/or KYN.

Example 4

GABA was measured by liquid chromatography with tandem mass spectrometry (HPLC-MS/MS). The supernatants obtained from the in vitro fermentations of Example 3 were subjected to centrifugation. Then, 200 μL of supernatant was transferred to 96-well plates. Components comprised in these supernatants were separated on an Acquity UPLC H-class system comprising degasser, quaternary pump, sample manager and thermostat column compartment equipped with TQDetector (Waters). The columns used were a UPLC BEH C18 guard column, 2.1×50 mm, 1.7 μM (Waters reference: 1866003975) and an Acquity UPLC BEH C18, 2.1×150 mm, 1.7 μM (Waters reference: 176001048). The eluents were: A 99.8% H₂O+0.2% formic acid; and B 99.8% ACN (LC-MS grade)+0.2% formic acid. Flow rate was 0.3 mL/min, column temperature was 40° C. The autosampler was set to 15° C., run time was 7.5 minutes. Gradient profile is shown in Table 5.

TABLE 5
LC flow rate parameters.

| Time (min) | Flow rate (mL/min) | % A |
|---|---|---|
| 0 | 0.3 | 95.0 |
| 0.25 | 0.3 | 95.0 |
| 2.70 | 0.3 | 85.0 |
| 4.00 | 0.3 | 60.0 |
| 5.00 | 0.3 | 0.0 |
| 5.20 | 0.3 | 0.0 |
| 6.00 | 0.3 | 0.0 |
| 6.10 | 0.3 | 95.0 |
| 7.20 | 0.3 | 95.0 |

As standards, GABA powder was used at a calibration range from 3 to 0.0003 μg/mL. The chromatographic injection volume was 2 μL, the syringe was washed after each injection. The MS method used was a multiple reaction monitoring (MRM) with a positive electrospray ionization. For GABA, the parent fragment m/z was 103.72 and the daughter fragment m/z was 85.85. The dwell time was 0.025 s, the cone voltage was 10 V, and the collision voltage was 13 V.

Kynurenine (KYN) quantification was performed by ELISA kit following the supplier's recommendation (FI-EM1862; Euromedex). Briefly, 50 μL of standard, blank or samples were added to 50 μL of Biotin-labeled Antibody Working Solution and incubated for 45 min at 37° C. The plate was washed three times with Wash Buffer, and 100 μL of HRP-streptavidin conjugate was added into each well. The plate was incubated for 30 minutes at 37° C. The plate was then washed five times with Wash Buffer. 90 μL of TMB substrate was added to each well followed by an incubation at 37° C. in dark for 10-20 minutes. Then, 50 μL of stop solution was added to each well and the absorbance was measured at 450 nm and correlated to a standard curve of known KYN concentrations.

### Example 5

The synthetic oligosaccharide preparations produced in Example 2 were individually subjected to swine microbiota in in vitro fermentations as described in Example 3, and analyzed as outlined in Example 4. The effect of contacting the microbiota with any one of the synthetic oligosaccharide preparations in terms of GABA production was calculated as log2 fold change of produced GABA compared to the log2 fold change of the GABA concentration obtained from the microbiota contacted with the glucose control, see Table 6. All of the synthetic oligosaccharide preparations comprising N-acetylglucosamine and/or condensed using methylphosphonic acid as catalyst were found capable of eliciting a significantly larger microbial production of GABA than the glucose control.

TABLE 6
Log2 fold changes in GABA production compared to a glucose
control (FC/Glu) by individual oligosaccharide preparations.

| sample/sugars | catalyst | FC/Glu |
|---|---|---|
| blank | | 0.97 |
| glucose control | | 1.00 |
| glucose + arabinose + lactose | methylphosphonic acid | 2.23 |
| glucose + xylose + N-acetylglucosamine | methylphosphonic acid | 2.61 |
| glucose + N-acetylglucosamine | methylphosphonic acid | 3.75 |
| glucose + xylose + arabinose | methylphosphonic acid | 1.97 |
| glucose + N-acetylglucosamine + lactose | (+)-camphor-10-sulfonic acid | 3.20 |
| glucose + sucrose + arabinose | methylphosphonic acid | 2.10 |
| glucose + lactose | methylphosphonic acid | 1.83 |
| glucose + xylose | methylphosphonic acid | 1.48 |
| glucose + sucrose + N-acetylglucosamine | methylphosphonic acid | 2.66 |
| glucose + fructose + N-acetylglucosamine | (+)-camphor-10-sulfonic acid | 2.00 |
| glucose + sucrose + N-acetylglucosamine | (+)-camphor-10-sulfonic acid | 1.69 |
| glucose + fructose + arabinose | methylphosphonic acid | 1.89 |
| glucose + arabinose | methylphosphonic acid | 1.60 |
| glucose + sucrose + mannose | methylphosphonic acid | 2.14 |
| glucose + fructose + N-acetylglucosamine | methylphosphonic acid | 2.43 |
| glucose + fructose + mannose | methylphosphonic acid | 1.76 |
| glucose + N-acetylglucosamine + mannose | methylphosphonic acid | 2.33 |
| glucose + sucrose + N-acetylglucosamine | phosphoric acid | 1.57 |
| glucose + N-acetylglucosamine | methylphosphonic acid | 1.92 |
| glucose + sucrose + lactose | methylphosphonic acid | 2.00 |
| glucose + sucrose + xylose | methylphosphonic acid | 1.53 |
| glucose + N-acetylglucosamine | (+)-camphor-10-sulfonic acid | 3.82 |
| glucose + fructose + lactose | methylphosphonic acid | 1.96 |
| glucose + N-acetylglucosamine | methylphosphonic acid | 4.06 |
| glucose + arabinose + N-acetylglucosamine | methylphosphonic acid | 2.46 |
| glucose + sucrose + N-acetylglucosamine | methylphosphonic acid | 1.86 |
| glucose + fructose + N-acetylglucosamine | methylphosphonic acid | 3.19 |
| glucose + fructose + xylose | methylphosphonic acid | 1.64 |
| glucose + fructose + N-acetylglucosamine | phosphoric acid | 1.66 |
| glucose + sucrose + N-acetylglucosamine | (+)-camphor-10-sulfonic acid | 2.13 |
| glucose + N-acetylglucosamine | (+)-camphor-10-sulfonic acid | 1.98 |
| glucose + N-acetylglucosamine | (+)-camphor-10-sulfonic acid | 1.71 |
| glucose + xylose + mannose | methylphosphonic acid | 1.74 |
| glucose + N-acetylglucosamine + lactose | methylphosphonic acid | 4.27 |
| glucose + arabinose + N-acetylglucosamine | phosphoric acid | 1.73 |
| glucose + sucrose + N-acetylglucosamine | methylphosphonic acid | 2.88 |
| glucose + xylose + lactose | methylphosphonic acid | 2.01 |
| glucose + arabinose + N-acetylglucosamine | (+)-camphor-10-sulfonic acid | 1.71 |

### Example 6

To demonstrate translatability of the in vitro results of the previous Examples to in vivo effects, a feeding trial in swine was performed for 14 days. To this end, five different treatments were tested, using twelve animals per treatment: As negative control, basal pig feed was used. As positive control, the same basal pig feed was supplemented with 30 g of GABA per ton of feed. Such GABA supplementation at 30 g/MT has been described to increase growth performance, to improve stress-related parameters and to lower biting incidences (Li et al. 2015. Can. J. Anim. Sci. 95:165-171; Bi et al. 2020. J Anim Physiol Anim Nutr. 104:590-596). Two test treatments were prepared by spraying either one of the oligosaccharide preparations prepared from glucose, sucrose and N-acetylglucosamine with (+)-camphor-10-sulfonic acid as catalyst, or from glucose and N-acetylglucosamine with methylphosphonic acid as catalyst (hereafter referred to as OP: A, or OP: B, respectively) onto the basal pig feed as mash. The oligosaccharide preparations were supplied in syrup form at a concentration of 70 brix, and added to the feed at an inclusion level of 1000 mg/kg. For the negative and the positive control, the same volume of water was sprayed on the basal pig feeds as mash, instead of the syrup of any of the test treatments.

The composition of the basal pig feed is shown in Table 7 below. Feeds were given for ad libitum consumption as mash during the experimental period. Phytase (Ronozme HiPhos) at 100 mg/kg feed was included in premix form using ground corn as carrier in the basal diet.

TABLE 7
Basal pig feed.

| | Experimental period |
|---|---|
| Ingredients (%) | Grower |
| Corn | 76.00 |
| Soybean meal | 19.60 |
| Soybean oil | 0.70 |
| NaCl | 0.35 |
| Premix vitamin-mineral 3144* | 0.30 |
| DL-Methionine | 0.05 |
| L-Lysine | 0.47 |
| L-Thr | 0.20 |
| L-Val | 0.12 |
| Limestone | 1.00 |
| Dicalcium phosphate | 1.20 |
| HiPhos 10 000 GT | 0.01 |

Calculated energy & nutrient contents

| | |
|---|---|
| Metabolizable energy (Kcal/kg) | 3303 |
| Crude protein (%) | 15.28 |
| Lysine (%) | 1.01 |
| Methionine (%) | 0.27 |
| Threonine (%) | 0.66 |
| Total P total (%) | 0.57 |
| Total Ca (%) | 0.68 |
| STTD P (%) | 0.37 |

*Premix vitamin mineral 3144 provided per kilogram of diet: Vitamin A 6510 I.U.; Vitamin E: 75 mg.; Vitamin K: 2 mg; Vitamin D3: 2007 IU; Vitamin B1: 0.99 mg; Vitamin B2: 5.02 mg; Vitamin B6: 2.01 mg; Vitamin B12: 0.03 mg; Pantothenic acid: 17.5 mg; Folic acid: 0.51 mg; Biotin 0.1 mg; Choline: 100 mg; Mn: 40 mg; Fe: 100 mg; Cu: 15 mg; Zn: 65 mg; I: 2 mg; Se: 0.4 mg.

Zootechnical parameters (mortality, weight gain, final weight, FCR-feed conversion ratio) were monitored. GABA and KYN were determined in plasma and feces samples; cortisol (COR), adrenocorticotrophic hormone (ACTH), serum neuropeptide Y (NPY), and lactate were determined in plasma samples.

Statistical analysis of performance: For this study the experimental unit was the pen, n=4 pigs/pens. The data was analyzed by a one-factor ANOVA followed by the Tukey multiple comparison test. The effects of treatment was considered significant at p<0.05. For analysis, JMP software (version16.0) was used. Statistical biological analysis: For GABA and KYN, and stress-related markers COR, ACTH, NPY, glucose/lactose, in this study, the experimental unit was pigs, n=12 pigs/treatment. The data was analyzed by a one-factor ANOVA followed by the Tukey multiple comparison test. The effects of treatment was considered significant at p<0.05. For analysis, JMP software (version 16.0) was used.

On day 12, the plasma GABA fold change compared to day 0 was 1.07 for the positive control; slightly increased with 1.12 for treatment with OP: A; and significantly increased with 1.73 for treatment with OP: B. Fold changes (compared to day 0) of plasma COR as stress indicator were found significantly decreased for treatment with OP: A and OP: B (0.27 and 0.38, respectively), compared to the fold changes of negative control and positive control (0.66, and 0.70, respectively). In agreement with these findings, fold changes (compared to day 0) of plasma NPY as stress-resistance indicator were found significantly increased for treatments with OP: A and OP: B (0.93 and 1.60, respectively), compared to the fold changes of negative control, and positive control (0.57, and 0.51, respectively). Thus, the scalability of the in vitro results to in vivo effects was found confirmed. As a further consequence of the determined differences in GABA and stress-related markers, fewer incidences of aggressive behavior such as tail biting could be observed, along with reduced stress, fear and anxiety, and improved animal performance in terms of weight gain and FCR.

### Example 7

In another feeding trial, 288 pigs were used in a study for 44 days, including a 28 days growing phase and a 16 days finishing phase. Six treatments were tested, using 48 animals per treatment. As negative control, basal diet feed was provided. Basal diet feeds are shown in Table 8 below. A GABA control was prepared by supplementing the basal diet feeds with 30 mg of GABA per kg of feed. In four test treatments, the oligosaccharide preparation prepared from glucose, N-acetylglucosamine and lactose using methylphosphonic acid as catalyst (hereafter referred to as OP: C) was added to the basal diet feeds at inclusion rates of either 50, 150, 250, or 500 mg of oligosaccharide preparation per kg of feed. To compensate for the mass difference between the treatment feeds comprising 500 mg of oligosaccharide preparation per kg of feed and the other treatment feeds, silica was added. In particular, 0.5 kg SiO₂ per 1000 kg of feed was added to treatment feeds of the negative control; 0.47 kg SiO₂ per 1000 kg of feed was added to treatment feeds of the GABA control in addition to the 0.03 kg of GABA per 1000 kg of feed; 0.45 kg, 0.35 kg and 0.25 kg of SiO₂ per 1000 kg of feed were added to the treatment feeds comprising 50, 150, and 250 mg of oligosaccharide preparation per 1000 kg of feed, respectively.

As in the previous example, feeds were given for ad libitum consumption as mash during the experimental period. Phytase Ronozme HiPhos at 100 mg/kg feed was included in the premix forms using ground corn as carrier in the basal diet (2*150 g). Feed main raw materials were milled in a hammer mill using a 4 mm sieve and mixed with the other ingredients (minerals, vitamins and amino acids) in 1500 L mixer. The glycan products were supplied in syrup form sprayed on the mash feed. The same volume of water was sprayed for negative control feeds.

TABLE 8
Basal diet feeds.

| | Experimental period | |
|---|---|---|
| Ingredients (%) | Grower | Finisher |
| Corn | 79.00 | 82.00 |
| Soybean meal | 19.60 | 14.00 |
| Soybean oil | 0.70 | 0.50 |
| NaCl | 0.35 | 0.35 |
| Premix vitamin-mineral 3144* | 0.30 | 0.00 |
| Premix vitamin-mineral 3145** | 0.00 | 0.30 |
| DL-Methionine | 0.06 | 0.06 |
| L-Lysine | 0.46 | 0.46 |
| L-Thr | 0.13 | 0.13 |
| L-Val | 0.07 | 0.07 |
| L-Trp | 0.12 | 0.12 |
| Limestone | 1.00 | 0.90 |
| Dicaclium phosphate | 1.20 | 1.10 |
| HiPhos 10 000 GT | 0.01 | 0.01 |

Calculated energy & nutrient contents

| | | |
|---|---|---|
| Crude protein (%) | 15.28 | 13.11 |
| Metabolizable energy (Kcal/kg) | 3303 | 3300 |
| Lysine (%) | 1.10 | 0.88 |
| Methionine (%) | 0.28 | 0.26 |
| Threonine (%) | 0.59 | 0.52 |
| Tryptophan (%) | 0.26 | 0.23 |
| Total P total (%) | 0.57 | 0.53 |
| STTD P (%) | 0.37 | 0.34 |
| Total CA (%) | 0.68 | 0.61 |

*Premix vitamin mineral 3144 provided per kilogram of diet: Vitamin A 6510 I.U.; Vitamin E: 75 mg.; Vitamin K: 2 mg; Vitamin D3: 2007 IU; Vitamin B1: 0.99 mg; Vitamin B2: 5.02 mg; Vitamin B6: 2.01 mg; Vitamin B12: 0.03 mg; Pantothenic acid: 17.5 mg; Folic acid: 0.51 mg; Biotin 0.1 mg; Choline: 100 mg; Mn: 40 mg; Fe: 100 mg; Cu: 15 mg; Zn: 65 mg; I: 2 mg; Se: 0.4 mg.
**Premix vitamin-mineral 3145 provided per kilogram of diet: Vitamin A: 15000 I.U .; Vitamin E: 100 mg .; Vitamin K: 20.0 mg; Vitamin C: 100 mg; Vitamin B1: 3 mg; Vitamin B2: 10 mg; Vitamin B6: 6 mg; Vitamin B12: 0.04 mg; Niacin: 60.0 mg; Pantothenic acid: 25.0 mg; Folic acid: 1.5 mg; Biotin 0.2 mg; Choline: 325 mg; Mn: 60 mg; Fe: 200 mg; Cu: 140 mg; Zn: 100 mg; 1: 2 mg; Se: 0.4 mg.

To study the effects of providing the animals with oligosaccharide preparations according to the invention, in particular with respect to animal welfare, a mixing challenge was conducted. Agonistic behavior after mixing of individual pigs amongst groups is known to be an animal welfare concern, resulting in skin lesions on the body, decreased growth performance and increased aggressive behavior. At the beginning of the trial, pigs were grouped by body weight. At day 28 all pigs were weighed before mixing, then one pig per pen was selected and exchanged with a pig of similar weight of same treatment group but from another pen. Performance data (weight, weight gain, FCR), social behavior and skin lesions were monitored. Metagenomic analyses were performed from feces samples. GABA, serotonin (5-HT), Trp (tryptophan) and KYN were analyzed from plasma and feces samples. COR, ACTH, NPY, chromogranin A (CgA) and haptoglobin (HPT) were analyzed from plasma samples. Statistics were performed as in the previous example.

In agreement with the previous examples, plasma GABA fold change could be found increased when oligosaccharide preparations of the invention were provided to the animals with the feed, compared to negative and GABA controls. Also plasma cortisol fold change could be found significantly decreased, and plasma 5-HT fold change significantly increased when oligosaccharide preparations of the invention were provided to the animals with the feed, compared to negative control. Average daily weight gain (ADG) between days 0-28 of the trial, i.e. prior to the mixing challenge, was 3.0% higher for the test treatment group compared to the negative control group: 862 g/d compared to 837 g/d, respectively. Mixing had a negative impact on ADG, as reflected by ADGs of 769 g/d and 810 g/d for days 28-42 for negative control and test treatment group, respectively. However, despite the stress imposed on the animals upon mixing, the test treatment groups receiving the oligosaccharide preparation had a 5.3% higher ADG than the negative control. When comparing the ADGs for days 0-42, the test treatment groups were significantly (P=0.04) higher (845 g/d) than the negative control group (814 g/d). Thus, feeding an oligosaccharide preparation according to the present invention was found to improve the animals' performance in terms of ADG in general, and in particular when the animals were confronted with stress. Average daily feed intake (ADFI) was not found to be significantly affected with 2004 g/day and 1991 g/day for days 0-28; 2577 g/day and 2605 g/day for days 28-42; and 2195 g/day and 2196 g/day for days 0-42 in the negative control group and the test treatment groups, respectively. Accordingly, the feed conversion ratio for days 0-28 was 3.3% decreased in the test treatment groups (2.32) compared to the negative control (2.40); and 1.1% decreased in the test treatment groups (3.32) compared to the negative control (3.36) for days 28-42; and significantly (P=0.07) decreased by 3.5% in the test treatment groups (2.60) compared to the negative control (2.70) for days 0-42. It was thus concluded, that energy from the feed consumed during stress was not converted to animal growth by animals of the negative control group, whereas animals having received the oligosaccharide preparation were less susceptible to stress and more successful in converting the energy from the feed to growth.

1. Method for modulating the level of at least one secondary metabolite (e.g. neutrotransmitter) in the body of an animal (e.g. increasing the level of gamma-aminobutyric acid (GABA) in the gastrointestinal tract of the animal; and/or increasing the ratio of kynurenine: tryptophan in the body of the animal; and/or increasing the ratio of peripheral serotonin: tryptophan in the digestive system of the animal; and/or increasing the ratio of melatonin: tryptophan in the digestive system of the animal; and/or decreasing the ratio of tryptamine: tryptophan in the digestive system of the animal), the method comprising one or more of the following steps:
administering an oligosaccharide preparation to the animal, wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
administering a probiotic composition to the animal, wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and/or
administering an enzyme composition to the animal, wherein the enzyme composition comprises a polypeptide comprising any one of the amino acid sequences of SEQ ID NOs: 1-493, or a functionally equivalent variant of said polypeptide.

2. Use (e.g. non-medical use) of an oligosaccharide preparation; and/or of a probiotic composition; and/or of an enzyme composition for modulating the level of at least one secondary metabolite (e.g. neutrotransmitter) in the body of an animal (e.g. increasing the level of gamma-aminobutyric acid (GABA) in the gastrointestinal tract of the animal; and/or increasing the ratio of kynurenine: tryptophan in the body of the animal; and/or increasing the ratio of peripheral serotonin: tryptophan in the digestive system of the animal; and/or increasing the ratio of melatonin: tryptophan in the digestive system of the animal; and/or decreasing the ratio of tryptamine: tryptophan in the digestive system of the animal), wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and
wherein the enzyme composition comprises a polypeptide comprising any one of the amino acid sequences of SEQ ID NOs: 1-493, or a functionally equivalent variant of said polypeptide.

3. Use (e.g. non-medical use) of an oligosaccharide preparation; and/or of a probiotic composition; and/or of an enzyme composition to improve animal performance (e.g. improve average body weight gain and/or reduce feed conversion ratio); and/or to improve animal welfare (e.g. reduce anxiety, stress and/or fear disorders),
wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and
wherein the enzyme composition comprises a polypeptide comprising any one of the amino acid sequences of SEQ ID NOs: 1-493, or a functionally equivalent variant of said polypeptide.

4. The method according to claim 1,
wherein the relative abundance of 4-aminobutyrate transaminase activity in the gastrointestinal tract of the animal is reduced;
wherein the relative abundance of glutamate decarboxylase activity in the gastrointestinal tract of the animal is increased;
wherein the relative abundance of gamma-glutamylputrescine synthetase (e.g. PuuA) activity in the gastrointestinal tract of the animal is increased;
wherein the relative abundance of NADP/NAD-dependent aldehyde dehydrogenase (e.g. PuuC) activity in the gastrointestinal tract of the animal is increased;
wherein the relative abundance of gamma-glutamyl-gamma-aminobutyrate hydrolase (e.g. PuuD) activity in the gastrointestinal tract of the animal is increased;
wherein the relative abundance of putrescine aminotransferase activity in the gastrointestinal tract of the animal is increased; and/or
wherein the relative abundance of gamma-aminobutyraldehyde dehydrogenase activity in the gastrointestinal tract of the animal is increased.

5. The method according to claim 1, wherein the level of GABA in the brain of the animal is increased at least 5% (e.g. at least 10%, 15%, 20%, 25%, 30%, 35%; or 10-35%) compared to the level of GABA in the brain of an animal not treated by the method, or not administered the oligosaccharide preparation, the probiotic composition and/or the enzyme composition.

6. The method according to claim 1, wherein the animal is selected from the group consisting of pet animals, dog, cat, canary, guinea pig, hamster, rabbit, mouse, rat, deer, boar, zoo animals, horse, donkey, poultry, swine, ruminant, chicken, cow, sheep, goat, pig, piglet, turkey, aquaculture, fish, shrimp, prawn, crayfish, crab, oyster, mussel, clam, trout, tilapia, salmon, carp, catfish, tuna, preferably, the animal is selected from the group consisting of poultry such as chicken, swine, ruminant such as cow.

7. The method according to claim 1, wherein the oligosaccharide preparation is comprised in a nutritional composition administered to an animal at an inclusion rate of at least 50 ppm (e.g. at least 50, 70, 100, 150, 200, 300, 400, 500 ppm).

8. The method according to claim 1, wherein n is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

9. The method according to claim 1, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

10. The method according to claim 1, wherein the oligosaccharide preparation has a weight average molecular weight from about 300 to 5000 g/mol (e.g. from about 2000 to 2800 g/mol, 2100 to 2700 g/mol, 2200 to 2600 g/mol, 2300 to 2500 g/mol, or 2320 to 2420 g/mol), 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

11. The method according to claim 1, wherein the relative abundance of oligosaccharides in each of the n fractions of the oligosaccharide preparation decreases monotonically with its degree of polymerization.

12. An oligosaccharide preparation; and/or a probiotic composition; and/or an enzyme composition for use in treatment, amelioration and/or prophylaxis of one or more disorders associated with an imbalanced level of GABA and/or an imbalanced ratio of kynurenine:tryptophan, serotonin:tryptophan, melatonin:tryptophan, and/or tryptamine:tryptophan,
wherein the one or more disorders are selected from the group of anxiety, stress, fear disorders, systemic inflammation, and local inflammation;
wherein the oligosaccharide preparation comprises at least n fractions of oligosaccharides each having a distinct degree of polymerization selected from 1 to n (DP1 to DPn fractions), wherein n is an integer greater than or equal to 2; wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry;
wherein the probiotic composition comprises at least one strain selected from Barnesiella sp. (e.g. Barnesiella viscericola, Barnesiella intestinhominis), and/or Bacteroides sp. (e.g. Bacteroides uniformis, Bacteroides cellulosilyticus, Bacteroides fragilis); and
wherein the enzyme composition comprises a polypeptide comprising any one of the amino acid sequences of SEQ ID NOs: 1-493, or a functionally equivalent variant of said polypeptide.
